(19) United States
(12) Patent Application Publication
(10) Pub. No.: US 2001/0010818 A1
(43) Pub. Date: Aug. 2, 2001

(54) Methods and formulations for reducing circulating antibodies

(76) Inventors: Steven B. Engle, Del Mar, CA (US); Richard M. Jack, Del Mar, CA (US); David S. Jones, San Diego, CA (US); Lin Yu, San Diego, CA (US)

Correspondence Address: MORRISON & FOERSTER LLP, 755 PAGE MILL RD, PALO ALTO, CA, 94304-1018, US

(21) Appl. No.: 09/766,365

(22) Filed: Jan. 18, 2001

Related U.S. Application Data
(63) Continuation of application No. 09/457,875, filed on Dec. 8, 1999, now abandoned.
(60) Provisional application No. 60/111,639, filed on Dec. 9, 1998.

(51) Int. Cl. A61K 31/70; A01N 43/04

(52) U.S. Cl. 424/140.1; 514/25

(57) ABSTRACT

The invention provides methods for reducing circulating levels of antibodies, particularly disease-associated antibodies. The methods entail administering effective amounts of epitope-presenting carriers to an individual. In other embodiments, ex vivo methods for reducing circulating levels of antibodies are provided which employ epitope-presenting carriers.

### RELATED APPLICATIONS

This application claims the priority benefit of provisional application U.S. Ser. No. 60/111,639, filed Dec. 9, 1998, the contents of which are incorporated by reference in their entirety.

1. Techical Field

This invention relates to reducing of circulating antibodies, particularly disease-associated antibodies, by binding the antibodies to an epitope-presenting carrier.

2. Background Art

There are many conditions and disorders which are associated with circulating antibodies of various classes. Some of these conditions are, for example, autoimmune disorders (such as lupus and idiopathic thrombocytopenia purpura) and IgE associated disorders.

One approach to treating, or combating, these disorders is to remove and/or reduce the circulating antibodies, even on a transient basis. One example of such an approach is apheresis, in which blood is removed from an individual and the antibodies are removed extracorporeally using an affinity column, such as a protein A column. See, e.g., U.S. Pat. Nos. 4,851,126, 4,255,627, 4,086,294, 5,147,290, 4,411,792; Badnarenko (1996) Clinics in Laboratory Medicine 16:907-929; Snyder et al. (1992) Blood 79:2237-245; Richter et al. (1993) Metabol. Clin. Exp. 42:888-894; Richter et al. (1997) ASAIO J. 43(1):53-59; Pascher et al. (1997) Transplantation 6363(6):867-875; Wallukat et al. (1996) Int'l J. Card. 54:191-195; Nillson et al. (1981) Blood 58:38-44; Watson et al. (1989) Cancer 64:1000; Suzuki et al. (1994) Autoimmunity 19:105-112; Suzuki et al. (1995) Artificial Organs 20(4):296-302; Bandarenko (1996) Clinics in Laboratory Medicine 16:907-929.

The problem with apheresis as a technique to reduce levels of circulating antibodies is that it is cumbersome and expensive. Further, apheresis often requires frequent and repetitive administrations.

Other literature describes inducing tolerance, i.e., reducing levels of circulating antibodies by inducing B cell anergy. See, e.g., U.S. Pat. Nos. 5,276,013, 5,391,785, 5,786,512, 5,726,329, 5,552,391, 5,268,454; PCT/US96/009976; PCT/US97/10075; PCT/US91/09176; U.S. Ser. No. 5,268,454; U.S. Ser. No. 08/118,055; U.S. Ser. No. 60/088,656; U.S. Ser. No. 60/103,088.

What is needed are improved methods of reducing circulating antibodies, particularly antibodies associated with disease.

All publications cited herein are hereby incorporated by reference in their entirety.

### DISCLOSURE OF THE INVENTION

The invention provides methods of reducing levels of circulating antibodies, particularly disease or disorder-associated antibodies.

Accordingly, in one aspect, the invention provides methods for reducing levels of circulating antibodies in an individual, particularly disease-associated antibodies, comprising administering to the individual an effective amount of an epitope-presenting moiety. The epitope-presenting moiety may be any of a number of embodiments, and is preferably a conjugate comprising a valency platform molecule and an epitope(s). The epitope may be any moiety, as long as it exhibits the requisite binding activity.

In another aspect, the invention provides ex vivo methods of reducing circulating antibodies using an eptiope-presenting moiety. The methods provide methods of reducing levels of disease-associated antibodies in an individual, comprising treating the individual's blood (including any component thereof which contains antibody) extracoporeally with an eptiope-presenting carrier under conditions that permit the antibodies to bind the epitope; removing antibody-eptiope-presenting carrier complexes, if any; and returning the blood to the individual.

### BRIEF DESCRIPTION OF THE DRAWINGS

FIG. 1 is a reaction scheme illustrating the enzymatic synthesis of the αGal epitope,2-[2-(2-thioethoxy) ethoxy]ethyl 3-O-(α-D-galactopyranosyl)-β-D-galactopyranoside.

FIG. 2 is a reaction scheme illustrating the chemical synthesis of the αGal epitope, 2-[2-(2-thioethoxy) ethoxy]ethyl 3-O-(α-D-galactopyranosyl)-β-D-galactopyranoside.

FIG. 3 is a reaction scheme illustrating the chemoenzymatic synthesis of the αGal epitope, ρ-aminophenyl 3-O-α-D-galactopyranosyl-α-D-galactopyranoside.

FIG. 4 illustrates two general synthetic strategies for conjugation chemistry.

FIG. 5 illustrates a third general synthetic strategy for conjugation chemistry.

FIG. 6 illustrates two dimeric platforms and four tetrameric platforms.

FIG. 7 illustrates four octameric platforms.

FIG. 8 illustrates a monomeric αGal conjugate and three dimeric αGal conjugates.

FIG. 9 illustrates two tetrameric αGal conjugates as described in Example 3.

FIG. 10 illustrates two tetrameric αGal conjugates as described in Example 3.

FIG. 11 illustrates two tetrameric conjugates of two αGal-isomers as described in Example 3.

FIG. 12 illustrates an octameric αGal conjugate as described in Example 3.

FIG. 13 illustrates an octameric αGal conjugate as described in Example 3.

FIG. 14 illustrates an octameric αGal conjugate as described in Example 3.

FIG. 15 illustrates an octameric αGal conjugate as described in Example 3.

FIG. 16 is a graph of OD550 versus fraction number and depicts the elution profile of anti-αGal from an αGal affinity column.

FIGS. 17A and 17B are graphs depicting affinity purified IgG (17A) and IgM (17B) anti-αGal binding to PK15 cells. Flow cytometric analysis results are shown as mean fluorescence intensity (MFI) versus dose, in μg/ml, of affinity-purified IgG or IgM (solid circles), or column flow-through IgG or IgM (open squares).

FIG. 18 is a graph depicting the effect of αGal valency of toleragens on inhibition of anti-αGal binding, measured by ELISA as described in Example 4. Symbols for inhibitors are as follows: open circles=αGal monomer; open squares, dashed lines=LJP 724 (trisaccharide monomer); open squares, dashed lines=LJP 725 (pentasaccharide monomer); solid circles, solid lines=αGal dimer; solid squares, solid lines=LJP 712 tetramer; solid triangles, solid lines=LJP 719 octamer; open triangles, dashed lines=LJP 728 (11-mer pentasaccharide-BSA); solid triangles, dashed lines=LJP 726 (11-mer trisaccharide [3-carbon linker]-HSA); solid squares, dashed lines=LJP 727 (11-mer trisaccharide [14-carbon linker]-HSA).

FIG. 19 is a bar graph of the percent plasma anti-αGal IgG following treatment with LJP 712, as described in Example 5.

FIG. 20 is a graph depicting activation of the classical complement pathway by various substances, as described in Example 5. Symbols are as follows: open circles, LJP 712; open squares, cobra venom factor (CVF); solid triangles, aggregated human gamma globulin (AHG). The dashed line represents results obtained with buffer alone.

FIG. 21 is a graph depicting activation of the alternative complement pathway by various substances, as described in Example 5. Symbols are as follows: open circles, LJP 712; open squares, CVF; solid triangles, AHG.

FIGS. 22A and 22B are graphs depicting the decrease in plasma anti-αGal IgG (22A) and IgM (22B) following treatment with octameric LJP 920 (cpd 46) (circles), compared with PBS (squares), as described in Example 5.

FIG. 23 is a graph comparing the effect of tetramer LJP 712 (open circles) with octamer LJP 920 (solid circles) on the percent plasma anti-αGal IgM, as described in Example 5. PBS (solid squares) was included as a negative control.

FIG. 24 depicts a strategy for the synthesis of compound 29.

FIG. 25 depicts a strategy for the synthesis of compounds 31 and 32.

FIGS. 26A and 26B depict the synthesis of compound 30.

### MODES FOR CARRYING OUT THE INVENTION

This invention provides effective methods of removing and/or reducing circulating levels of antibodies, particularly disease-associated antibodies. This removal and/or reduction is generally transient, as it is based on binding to circulating antibodies as opposed to causing B cell anergy, although induction of B cell anergy may accompany these methods.

General Techniques

The practice of the present invention will employ, unless otherwise indicated, conventional techniques of molecular biology (including recombinant techniques), microbiology, cell biology, biochemistry and immunology, which are within the skill of the art. Such techniques are explaned fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

Definitions

For purposes of this invention, “reducing” and/or “removing” circulating antibodies means that the level of free, or unbound, circulating antibodies has been reduced. In some embodiments, by binding of epitope-presenting carrier to antibody, antibody is prevented from being an effector molecule, i.e., binding other targets, and is thus “reduced.” In some embodiments, “reducing” circulating antibodies includes clearance of antibody, e.g., physical removal from circulation. One way this way this clearance is effected is clearance of a complex comprising an epitope-presenting carrier and antibody by reticuloendothelial system.

An “epitope” is a term well-understood in the art and means any chemical moiety which exhibits specific binding to an antibody. An “epitope” can also comprise an antigen, which is a moiety that contains an epitope, and, as such, also specifically binds to antibody.

An epitope or antigen that “specifically binds” to an antibody is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances.

An “antibody” (interchangeably used in plural form) is an immunoglobulin molecule capable of specific binding to a target, such as a carbohydrate or polypeptide, through at least one antigen recognition site, located in the variable region of the immunoglobulin molecule. As used herein, the term encompasses not only intact antibodies, but also fragments thereof (such as Fab, Fab′, F(ab′)₂, Fv), single chain (ScFv), mutants thereof, fusion proteins comprising an antibody portion, humanized antibodies, and any other modified configuration of the immunoglobulin molecule that comprises an antigen recognition site of the required specificity.

As is well understood by those skilled in the art, a “disease-associated antibody” is an antibody whose production occurs during a disease state and/or whose production is undesireably, such as in autoimmune diseases and transplantation rejection. Examples of disease-associated antibodies are known in the art and include, but are not limited to, anti-double-tranded DNA antibodies (lupus) and anti-αGal antibodies (transplantation rejection).

“Naturally occurring” refers to an endogenous chemical moiety, such as a carbohydrate, polynucleotide or polypeptide sequence, i.e., one found in nature. Processing of naturally occurring moieties can occur in one or more steps, and these terms encompass all stages of processing. Conversely, a “non-naturally occurring” moiety refers to all other moieties, i.e., ones which do not occur in nature, such as recombinant polynucleotide sequences and non-naturally occurring carbohydrates.

As used herein, the term “mimetic” (also termed an “analog”) means a biological or chemical compound which specifically binds to an anti-αGal antibody. As such, for purposes of this invention, an “αGal epitope” includes mimetics of naturally-occurring αGal (such as peptides). A “mimetic” shares an epitope, or binding specificity, with αGal. A mimetic may be any chemical substance which exhibits the requisite binding properties, and thus may be, for example, a simple or complex organic or inorganic molecule; a polypeptide; a polynucleotide; a carbohydrate; a lipid; a lipopolysaccharide; a lipoprotein, or any combination of the above, including, but not limited to, a polynucleotide-containing polypeptide; a glycosylated polypeptide; and a glycolipid. The term “mimetic” encompasses the term “mimotope”, which is a term well known in the art.

An “individual” is a vertebrate, preferably a mammal, more preferably a human. Mammals include, but are not limited to, farm animals, sport animals, pets, primates, mice and rats.

An “effective amount” is an amount sufficient to effect beneficial or desired results, including clinical results. An effective amount can be administered in one or more administrations. For purposes of this invention, an effective amount is an amount sufficient to reduce circulating levels of free antibodies (i.e., unbound to epitope on carrier).

A “carrier” is a molecule which contains attachment sites for epitope(s). One preferred example of a carrier is a valency platform molecule. The terms “carrier” and “moiety” are used interchangeably herein.

An “epitope presenting carrier” is a carrier which contains attached, or bound, epitopes, at least some of which (at least two of which) are able to bind to an antibody of interest.

As used herein “valency platform molecule” means a nonimmunogenic molecule containing sites which allow the attachment of a discrete number of epitopes and/or mimetic(s) of epitopes. A “valency” of a conjugate or valency platform molecule indicates the number of attachment sites per molecule for an epitope(s). Alternatively, the valency of a conjugate is the ratio (whether absolute or average) of epitope to valency platform molecule.

“Nonimmunogenic”, when used to describe a carrier (including a valency platform molecule), means that the carrier (such as a valency platform molecule) fails to elicit an immune response (i.e., T cell and/or B cell response), and/or fails to elicit a sufficient immune response, when it is administered by itself to an individual. The degree of acceptable irumune response depends on the context in which the valency platform molecule is used, and may be empirically determined.

An epitope that is “conjugated” to a carrier (such as a valency platform molecule) is one that is attached to the carrier, either by covalent and/or covalent interactions.

A “stable complex” is one that sufficiently persists after its formation to allow subsequent detection and/or removal.

A “T cell epitope” means a component or portion thereof for which a T cell has an antigen-specific specific binding site, the result of binding to which activates the T cell. Where an embodiment of the invention is described as “lacking” a T cell epitope, this is taken to mean that a T cell epitope is not detectable using standard assays in the art. For purposes of this invention, an epitope that “lacks” a T cell epitope means that the epitope lacks a T cell epitope which causes T cell activation in the individual(s) to be treated (i.e., who is to receive an epitope-presenting carrier. It is likely that, for example, an epitope may lack a T cell epitope(s) with respect to an individual, or a group of individuals, while possessing a T cell epitope(s) with respect to other individual(s). Methods for detecting the presence of a T cell epitope include assays which detect T cell proliferation (such as thymidine incorporation). Polypeptides or other antigens that fail to induce statistically significant incorporation of thymidine above background (i.e., generally p less than 0.05 using standard statistical methods) are generally considered to lack T cell epitopes, although it will be appreciated that the quantitative amount of thymidine incorporation may vary, depending on the polypeptide (or other antigen) being tested. Generally, a stimulation index below about 2-3, more preferably less than about 1, indicates lack of T cell epitopes. The presence of T cell epitopes can also be determined by measuring secretion of T cell-derived lymphokines according to standard methods. Location and content of T cell epitopes, if present, can be determined empirically.

The term “blood” as used herein is bodily fluid including a cellular component and plasma. “Blood” means whole blood or a component thereof. Treating an “individual's blood” means that any or all of an individual's blood is treated.

A “stable complex” is one that sufficiently persists after its formation to allow subsequent detection and/or removal.

Methods of the invention

The invention provides methods of reducing circulating levels of antibodies, particularly disease-associated antibodies. These methods generally comprise administering an effective amount of an epitope-presenting (which includes an antigen-presenting) carrier (or a composition comprising an epitope-presenting carrier) to an individual. These methods are especially useful for effecting safe (i.e., lack of inflammation and/or other undesirable side effects) and rapid clearance in a more simple and desirable way than using other methods known in the art, such as apheresis. Without wishing to be bound by a particular theory, the inventors note that, by binding antibody on multivalent carrier, sufficiently large complexes are likely formed which may effect rapid clearance from circulation without formation of excessively large multi-valent complexes which would cause undesired side effects, such as inflammation, although, as noted below, “reduction” of circulating antibody levels does not require this clearance for purposes of this invention. The initial reduction of circulating antibody is due to binding the epitope-presenting carrier, and further more effective reduction is obtained by clearance.

For purposes of this invention, the reduction and/or removal of antibody is preferably selective, i.e., only that antibody for which reduction or removal is desired is affected. However, it may be acceptable that larger classes of antibodies are removed which include the antibody of interest. The extent of cross-reactivity of binding by the epitope will generally govern the types of antibody that are removed from circulation. Preferably, the reduction (as reflected, for example, by titer) is preferably at least about 50%, preferably at least about 60%, preferably at least about 80%, preferably at least about 85%, preferably at least about 90%, preferably at least about 95%. It is understood that, for purposes of this invention, total reduction (i.e., 100%) need not be effective in order for these methods to be efficacious. Methods of measuring antibody titer, either by binding or neutralizing assays, are well known in the art.

It is also understood that reduction and/or removal of particular sub-classes of antibodies may be desirable, and that not all classes of epitope binding antibodies need be reduced and/or removed. For example, because IgM antibodies mediate acute rejection in xenotransplantation, reduction of this class of αGal antibodies may be indicated. For example, we have found that, in the case of αGal antibodies, varying the valency may affect, and can have a significant impact, on adsorbing (binding) particular classes of antibody, as described in the Example section.

In some embodiments, methods are provided for reducing levels of circulating disease-associated antibodies in an individual, comprising administering to the individual an effective amount of an epitope presenting carrier (or composition comprising an epitope-presenting carrier) comprising a plurality of epitopes conjugated to a carrier which presents the epitopes in a manner effective to adsorb (i.e., bind) the antibodies.

The epitope presenting carrier is multivalent, i.e., is capable of presenting more than one epitope. Preferably, the valency is at least two. In other embodiments, the valency is at least three, at least four, at least six, at least eight, at least 10, at least 12, at least 16, at least 20, at least 24, at least 30, at least 32, at least 36, at least 40, at least 42, at least 46, at least 50. The upper limit of the valency is not necessarily critical, as long as the epitope-presenting carrier effects reduction and/or clearance without undesirable side effects.

In some embodiments, the valency is two. In other embodiments, the valency is four. In other embodiments, the valency is six. In other embodiments, the valency is any of the following: eight; 10; 12; 16; 20; 22; 24; 26; 28; 30; 32; 34; 36; 38; 40; 42; 44; 46; 48; 50; 52; 54; 56; 58; 60; 62; 64; 66; 68; 70; and increments of two, until 128.

Epitope-presenting carriers

Any of a variety of carriers may be used, as long as the carrier does not elicit an undesirable or unacceptable immune response. The carrier may be any chemical moiety, and have any chemical structure, including, but not limited to, organic and inorganic molecules, polypeptides (i.e., polymers of amino acids), nucleic acids, carbohydrates, other polymers, artificial structures, and lipid structures (such as liposomes or micelles) made by standard techniques, or polymerized as described in U.S. Pat. No. 5,512,294.

A carrier may be proteinaceous or non-proteinaceous (i.e., organic). Examples of proteinaceous platforms include, but are not limited to, albumin, gammaglobulin, immunoglobulin (IgG) and ovalbumin. Borel et al. (1990) Immunol. Methods 126:159-168; Dumas et al. (1995) Arch. Dematol. Res. 287:123-128; Borel et al. (1995) Int. Arch. Allergy Immunol. 107:264-267; Borel et al. (1996) Ann. N.Y. Acad. Sci. 778:80-87.

Preferably, the epitope-presenting carriers are conjugates which comprise a chemically defined valency platform molecule in which a precise valency (as opposed to an average) is provided. See, for example, commonly owned U.S. Pat. Nos. 5,162,515; 5,276,013; 5,552,391; 5,391,785; 5,786,512; 5,726,329; 5,268,454; 5,606,047; and 5,663,395. See also commonly-owned U.S. Ser. Nos. 08/482,651; 08/660,092; 08/760,508. Accordingly, a defined valency platform is a platform with defined structure, thus a defined number of attaclunent points and a defined valency. In contrast to previously described, more traditional platforms, these platforms have the advantage of having a homogeneous (i.e., uniform) molecular weight (as opposed to polydisperse molecular weight), and are thus “chemically defined”. Accordingly, it is understood that a population of conjugates using these platforms comprise a platform of homogeneous molecular weight or are substantially monodisperse (i.e., have a narrow molecular weight distribution). A measure of the breadth of distribution of molecular weight of a sample (such as a composition and/or population of platform molecules) of a platform molecule is the polydispersity of the sample. Polydispersity is used as a measure of the molecular weight homogeneity or nonhomogeneity of a polymer sample. Polydispersity is calculated by dividing the weight average molecular weight (Mw) by the number average molecular weight (Mn). The value of Mw/Mn is unity for a perfectly monodisperse polymer. Polydispersity (Mw/Mn) is measured by methods available in the art, such as gel permeation chromatography. The polydispersity (Mw/Mn) of a sample of platform molecules is preferably less than 2, more preferably, less than 1.5, or less than 1.2, less than 1.07, less than 1.02, or, e.g., about 1.05 to 1.5 or about 1.05 to 1.2. Typical polymers generally have a polydispersity of 2-5, or in some cases, 20 or more. Advantages of the low polydispersity property of the valency platform molecules include improved biocompatibility and bioavailability since the molecules are substantially homogeneous in size, and variations in biological activity due to wide variations in molecular weight are minimized. The low polydispersity molecules thus are pharmaceutically optimally formulated and easy to analyze. Further there is controlled valency of the population of molecules in the sample.

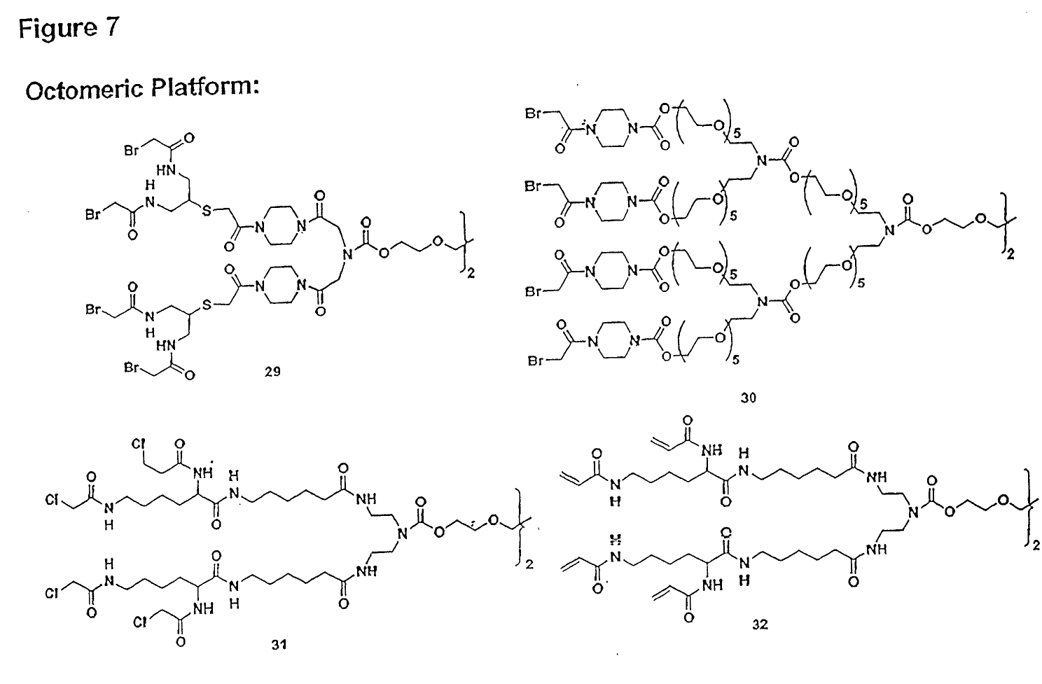

In some embodiments, the valency platform molecule is a carbamate, i.e., —O—C(=O)—N<). See U.S. Ser. No. 60/111,641 (“Valency Platform Molecules Comprising Carbamate Linkages”). An example of a carbamate platform is compound 30, FIG. 7.

Preferred valency platform molecules are biologically stabilized, i.e., they exhibit an in vivo excretion half-life often of hours to days to months to confer therapeutic efficacy, and are preferably composed of a synthetic single chain of defined composition. They generally have a molecular weight in the range of about 200 to about 200,000, preferably about 200 to about 50,000 (or less, such as 30,000). Examples of valency platform molecules within the present invention are polymers (or are comprised of polymers) such as polyethylene glycol (PEG), poly-D-lysine, polyvinyl alcohol, polyvinylpyrollidone, D-glutamic acid and D-lysine (in a ratio of 3:2). Preferred polymers are based on polyethylene glycols (PEGS) having a molecular weight of about 200 to about 8,000. Other suitable platform molecules for use in the conjugates of the invention are albumin and IgG.

Preferred valency platform molecules suitable for use within the present invention include the chemically-defined, non-polymeric valency platform molecules disclosed in co-owned U.S. Pat. No. 5,552,391. Particularly preferred homogeneous chemically-defined valency platform molecules suitable for use within the present invention are derivatized 2,2′-ethylenedioxydiethylamine (EDDA) and triethylene glycol (TEG).

Additional suitable valency platform molecules include, but are not limited to, tetraaminobenzene, heptaaminobetacyclodextrin, tetraaminopentaerythritol, 1,4,8,11-tetraazacyclotetradecane (Cyclam) and 1,4,7,10-tetraazacyclododecane (Cyclen).

In other embodiments, the tetra-bromoacetyl platform PIZ/IDA/TEG platform is used. Derivatives of the PIZ/IDA/TEG (PITG) platform can be prepared as shown below. See PCT/US97/10075 and PCT/US96/09976 for other examples of suitable platforms.

Examples of Compatible Cross-linking Groups on PITG Platform

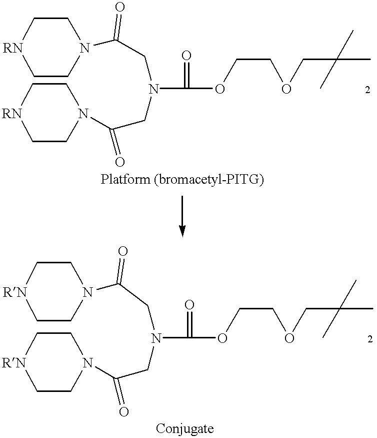

| Platform | Epitope | Conjugate |
|---|---|---|
| R = XCH₂CO | D1-SH | R′ = D1-SCH₂CO |

By way of example of a conjugate embodiment used in the methods of this invention, an polypeptide epitope is prepared with a thiol linker at the N terminus by chemical or enzymatic synthesis, or by recombinant methods. The linker can be cysteine or an SH containing moiety The modified epitope may then be alkylated by a suitably derivatized platform (such as bromoacetyl or iodoacetyl).

In general, the above-described platforms are made by standard chemical synthesis techniques. PEG must be derivatized and made multivalent, which is accomplished using standard techniques. Some substances suitable for conjugate synthesis, such as PEG, albumin, and IgG are available commercially.

In other embodiments, valency platforms may be used which, when conjugated, provide an average valency (i.e., these platforms are not chemically defined in terms of their valency). Examples of such platforms are polymers such as linear PEG; branched PEG; star PEG; polyamino acids, such as DEK; polylysine; proteins; amino-functionalized soluble polymers.

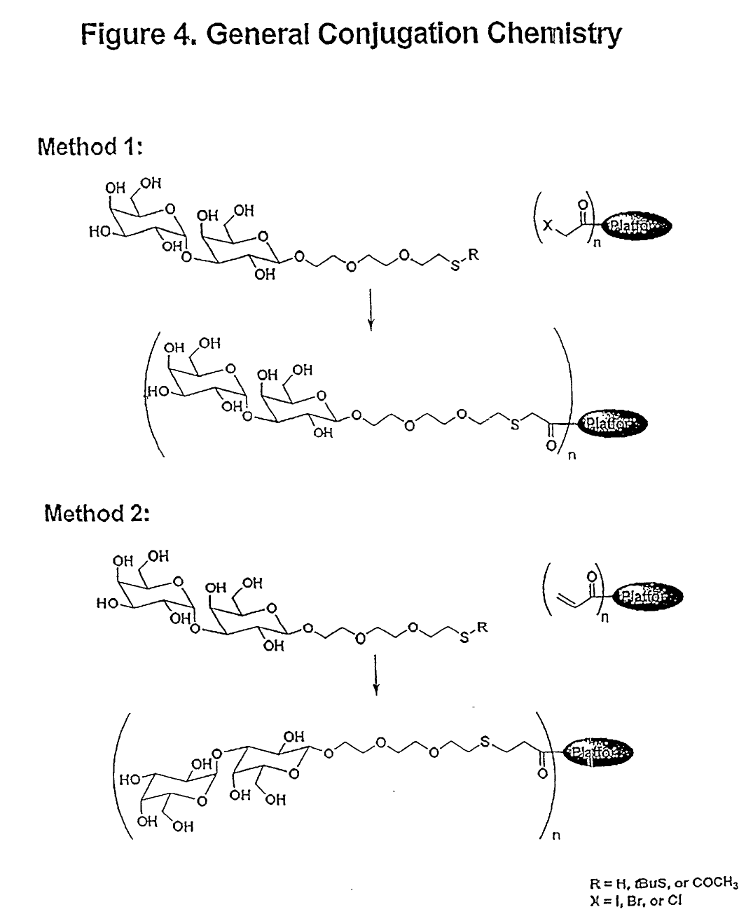

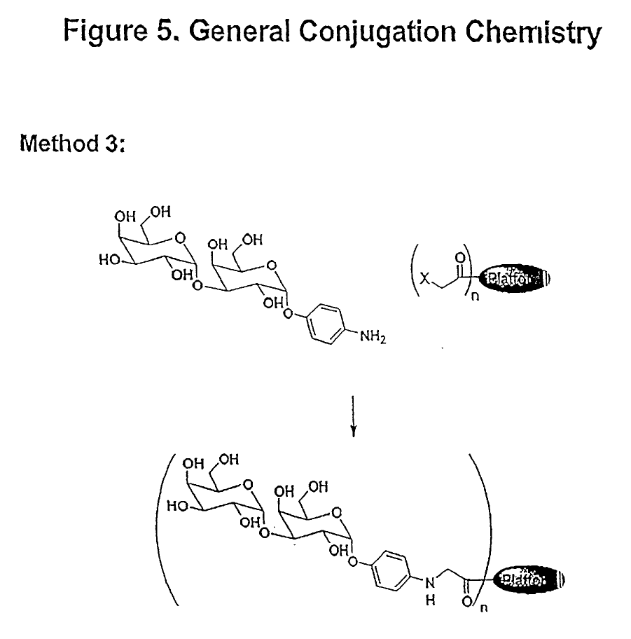

Covalent conjugation of epitope(s) with a carrier such as a valency platform molecule is generally performed using standard chemical techniques. The following are examples of standard chemistry which can be used: 1) thiol substitution; 2) thiol Michael addition; 3) amino alkylation; 4) disulfide bond formation. FIGS. 4 and 5 provide general, exemplary conjugation strategies.

Conjugation of an epitope to a valency platform molecule may be effected in any number of ways, typically involving one or more crosslinking agents and functional groups on the epitope and valency platform molecule. Platforms and epitope(s) must have appropriate linking groups. Linking groups are added to platforms using standard synthetic chemistry techniques. Linking groups may be added to an αGal epitope(s) using either standard solid phase synthetic techniques or recombinant techniques (if, for example, the αGal epitope is a peptide). Recombinant approaches may require post-translational modification in order to attach a linker, and such methods are known in the art.

As a further example, if the epitope is a polypeptide, polypeptides contain amino acid side chain moieties containing functional groups such as amino, carboxyl, or sulfhydryl groups that serve as sites for coupling the polypeptide to the platform. Residues that have such functional groups may be added to the polypeptide if the polypeptide does not already contain these groups. Such residues may be incorporated by solid phase synthesis techniques or recombinant techniques, both of which are well known in the peptide synthesis arts. When the polypeptide has a carbohydrate side chain(s), functional amino, sulfhydryl and/or aldehyde groups may be incorporated therein by conventional chemistry. For instance, primary amino groups may be incorporated by reaction with ethylenediamine in the presence of sodium cyanoborohydride, sulfihydryls may be introduced by reaction of cysteamine dihydrochloride followed by reduction with a standard disulfide reducing agent, while aldehyde groups may be generated following periodate oxidation. In a similar fashion, the valency platform molecule may also be derivatized to contain finctional groups if it does not already possess appropriate functional groups.

Hydrophilic linkers of variable lengths are useful for connecting epitopes to valency platform molecules. Suitable linkers include linear oligomers or polymers of ethylene glycol. Such linkers include linkers with the formula R¹S(CH₂CH₂O)ₙCH₂CH₂O(CH₂)ₘCO₂R² wherein n=0-200, m=1 or 2, R¹= H or a protecting group such as trityl, R²=H or alkyl or aryl, e.g., 4-nitrophenyl ester. These linkers are useful in connecting a molecule containing a thiol reactive group such as haloaceyl, maleiamide, etc., via a thioether to a second molecule which contains an amino group via an amide bond. These linkers are flexible with regard to the order of attachment, ie., the thioether can be formed first or last.

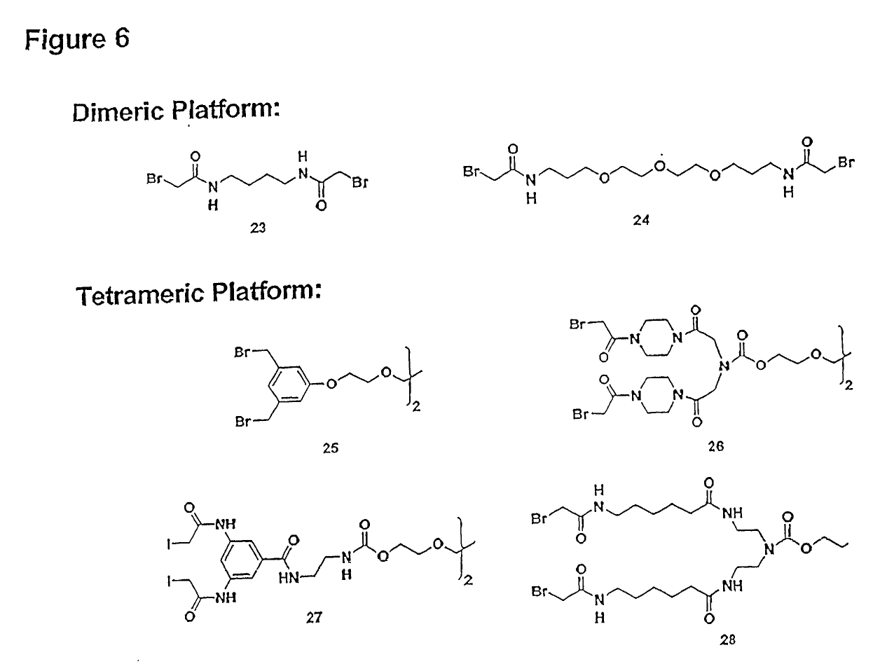

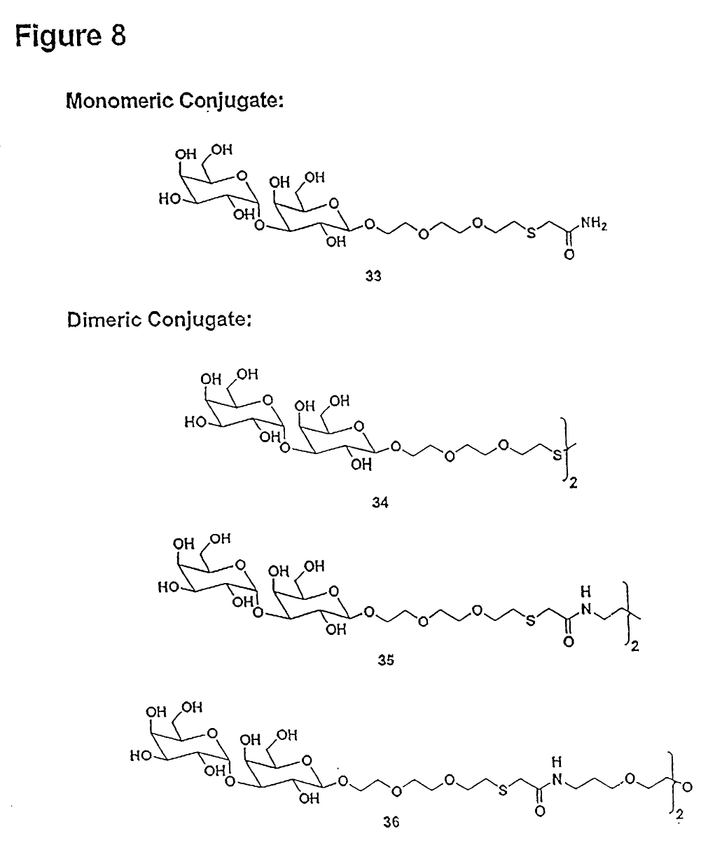

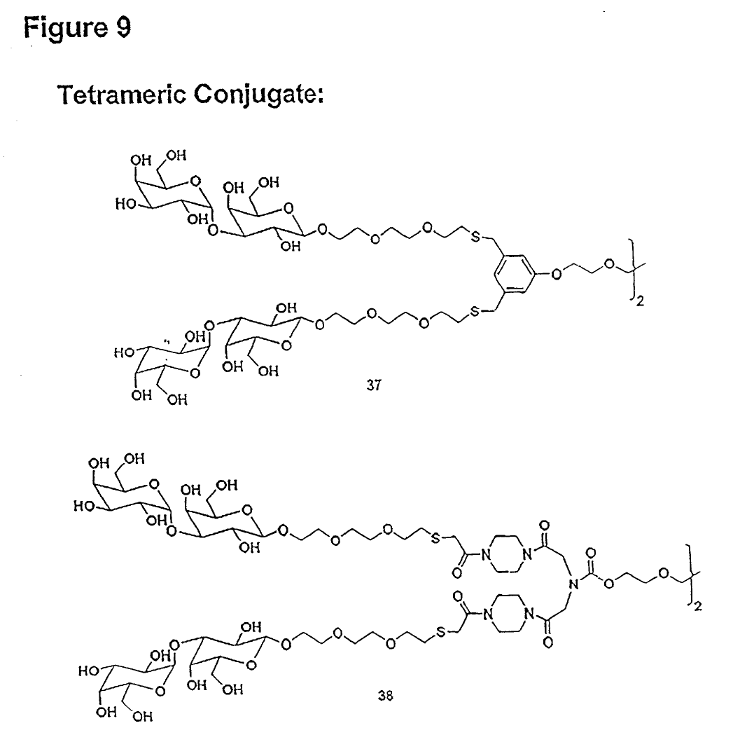

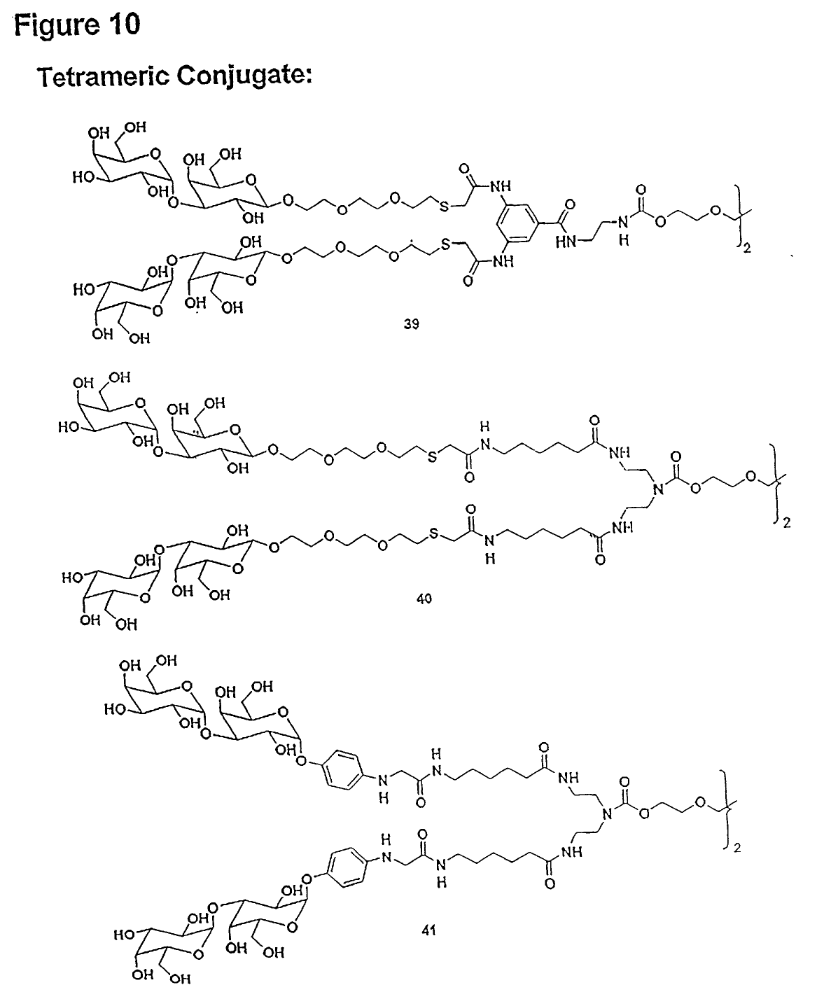

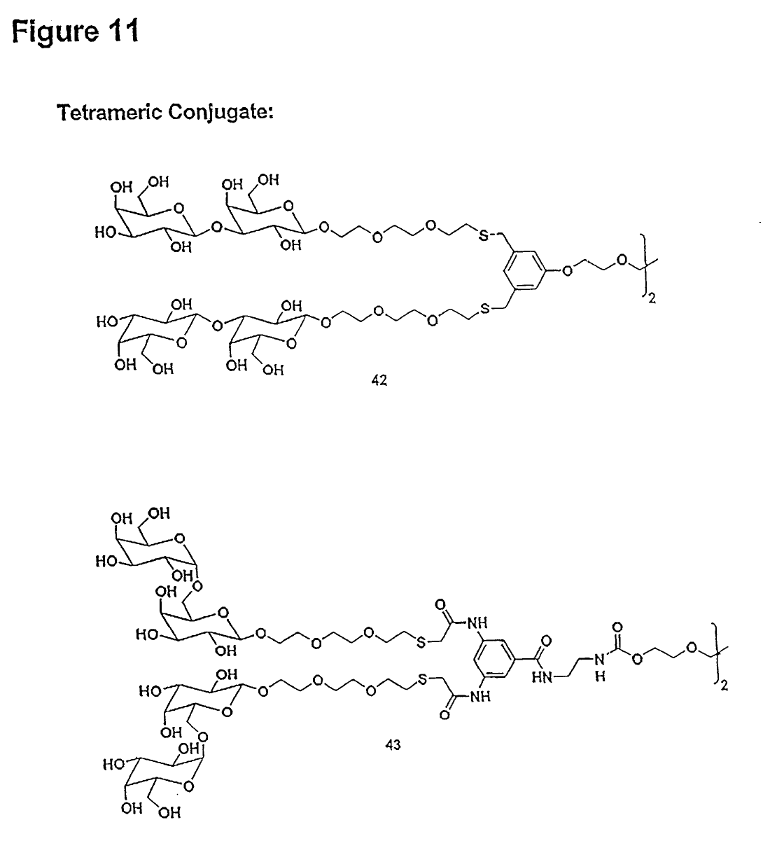

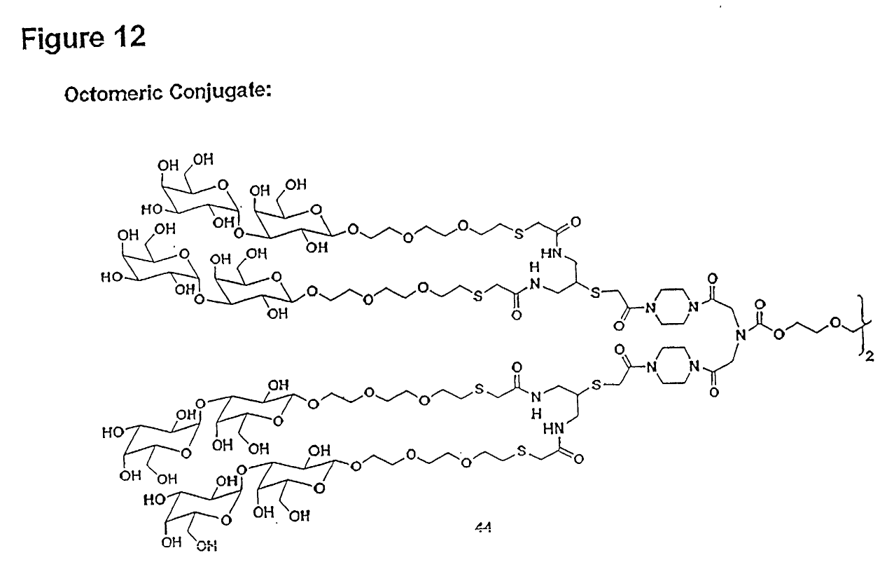

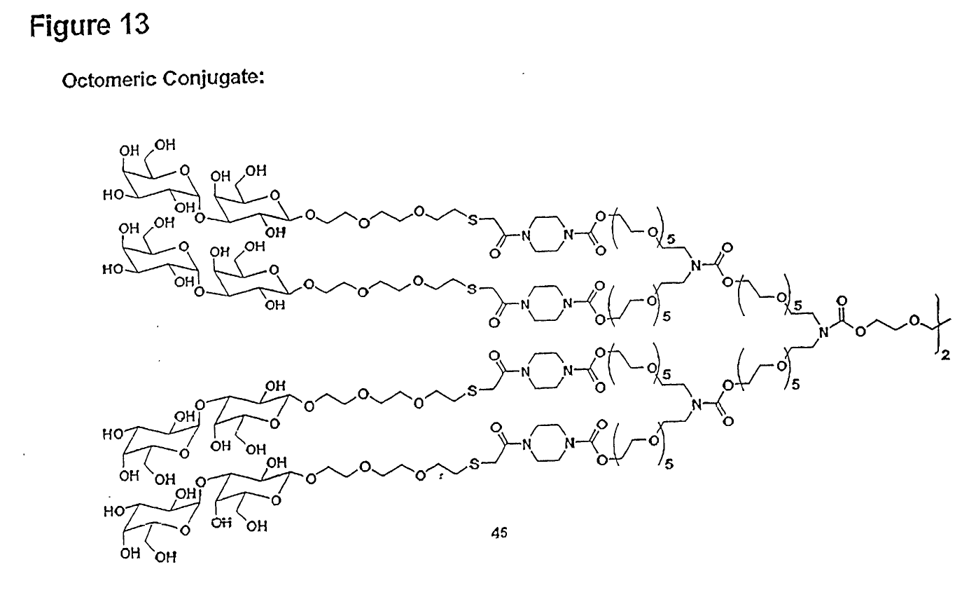

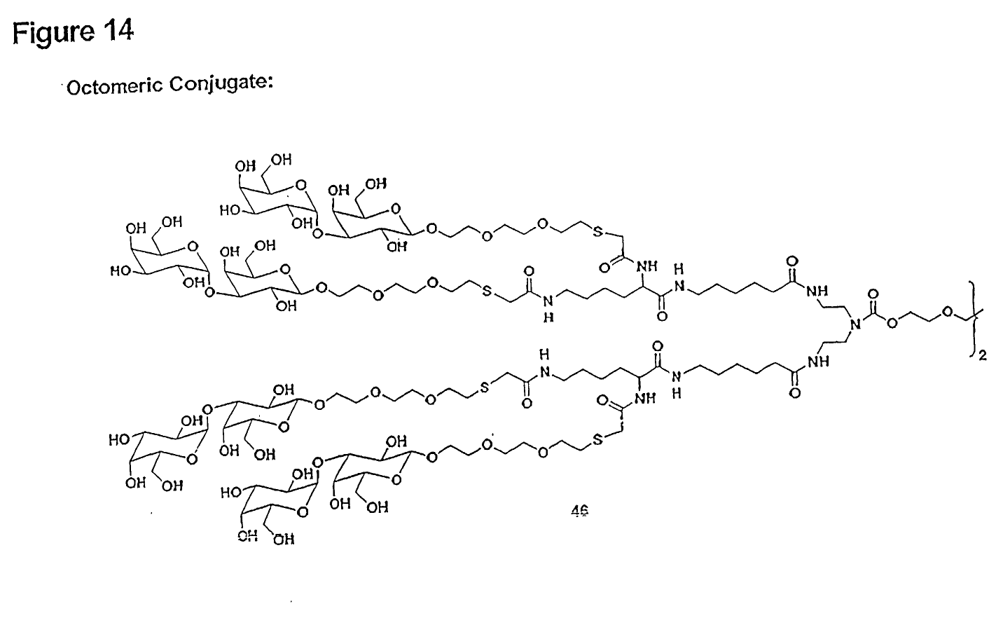

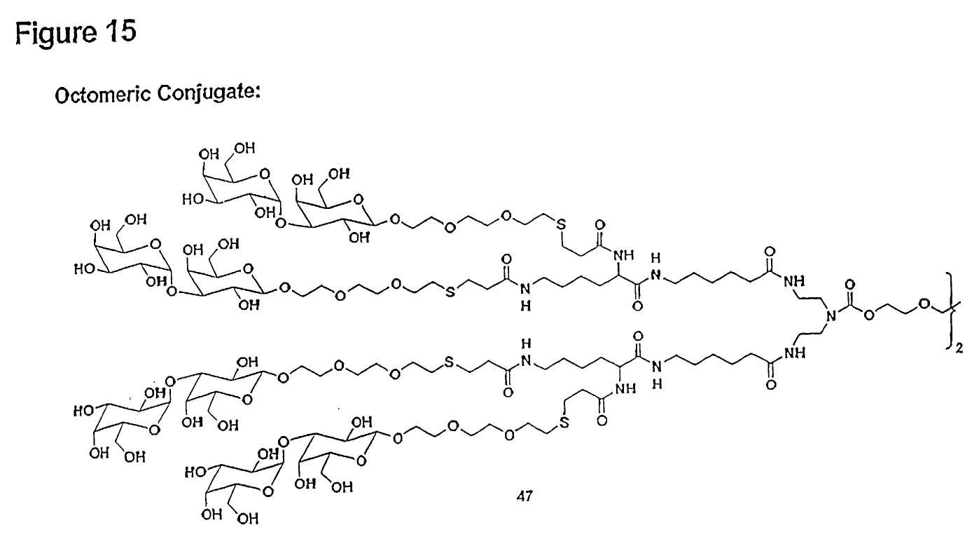

Particular conjugates are described in Example 2 and are depicted in FIGS. 6-15, which accordingly are provided as embodiments of the invention.

While the above discussion has exemplified preferred embodiments, namely those which employ valency platform molecules, it is clear that the carriers used in the invention may be any of a number of other moieties, and that many of the principles described above would likewise apply to other types of carriers.

As an example, liposomes may be used. Liposome technology is known in the art and need not be described in detail herein. As a brief summary, epitopes are appropriately inserted (i.e., inserted so that the binding moiety is available to bind to antibody, which may involve attaching “tails” to the epitope(s) for insertion) into liposomes, which may be of varying size. Mahato et al. (1997) Pharm. Res. 14:853-859. Liposomal preparations include, but are not limited to, cytofectins, multilamellar vesicles and unilamellar vesicles. Polymeric liposomes are described in U.S. Pat. No. 5,512,294.

As another example, multiple antigen peptides (MAPs) may be used. Posnett et al. (1988) J. Biol. Chem. 263:1719-1725; Tam (1989) Methods Enz. 168:7-15. MAPs have a small immunologically inert core having radially branching lysine dendrites, onto which polypeptide epitopes may be anchored. MAPs may be synthesized using methods known in the art, for example, a solid-phase methods, such as that described in Merrifield et al. (1963) J. Am. Chem. Soc. 85:2149.

Epitopes

Any epitope, or antibody-binding moiety, can be used. An epitope may be a polypeptide, organic, or inorganic molecule. Such antibody-binding moieties are known in the art and/or may be developed using standard methods and assays in the art, such as antibody binding assays. Antibodies used for assays to test and/or develop epitopes may be also obtained using standard assays in the art, such as affinity purification (or in some cases may be commercially available). Assays that may be used to determine whether a putative epitope or antigen exhibits requisite binding activity include, but are not limited to, filamentous phage random peptide libraries and screening (by, for example, biopanning, micropanning, phage-capture ELISA, phage-ELISA, colony blot, peptide ELISA, competitive binding peptide ELISA).

Examples of suitable epitopes include, but are not limited to, those that bind to: lupus anti-DNA antibodies (see U.S. Pat. Nos. 5,162,515; 5,391,785; 5,276,013; 5,786,512; 5,726,329; 5,552,391, 5,268,454); anti-galactose alpha 1,3 galactosyl (αGal) antibodies; anti-cardiolipin antibodies; antiphospholipid antibodies; IgE antibodies; anti-factor VIII antibodies; anti-factor IX antibodies; anti-β₂GPI antibodies, particularly to domain 1; anti-platelet antibodies; antibodies associated with idiopathic thrombocytopenia purpura (ITP); anti-adenovirus antibodies (which may be problematic when adenovirus is administered as a therapeutic agent); anti- adeno-associated virus (AAV) antibodies (which may be problematic when AAV is administered as a therapeutic agent); anti-alpha chain acetyl choline receptor (myasthenia gravis); anti-RhD antigen antibodies (i.e., Rh disease); anti-thyroid antibodies (for example, in auto-immune thyroiditis).

Of particular interest are epitopes that bind any undesired blood group antibody that may interfere with allo- or xenotransplantation. For example, antibodies that bind the structure Galα1-3Galβ1-4GlcNAcβ1-3Galβ1-4Glcβ1- can be removed using αGal epitopes described elsewhere in this application. In a similar fashion, epitopes can be designed that antigenically resemble the antigenic site on polysaccharides and are suitable for removing antibodies against other carbohydrate-based blood group antigens, including but not limited to those of the ABO system, the MN system, the Lewis system, and the Bombay phenotype.

Also of interest are epitopes that bind the anti-polynucleotide (particularly anti-double stranded DNA) antibodies that occur in systemic lupus erythematosis. Preferred platforms for such epitopes are tetrabromoacetyl compounds, and other tetravalent and octavalent valency platform molecules. D. Jones et al. (1995) J. Med Chem. 38:2138-2144; and U.S. patent references provided above. We have observed that administration of such lupus conjugates in humans have resulted in reduction of circulating anti-ds DNA antibodies. Jones (1995); Weisman et al. (1997) J. Rheum. 24:314-318; Iverson et al. (1998) Lupus 7 (Suppl. 2): S166-S169.

The suitability of particular epitopes for removing antibodies according to this invention can be confirmed empirically. For example, to select the optimum epitope from a library of small drug molecules believed to mimic the immunogenic epitope for a particular autoimmune disease, a family of carriers can be constructed in which each of the candidates is alternatively displayed on a similar carrier molecule or platform. The composition is then tested for efficacy. For example, for in vivo use, an animal model is used in which there are circulating antibodies of the undesired type. The animals can be immunized with an appropriate antigen to initiate the antibody response, if necessary. Test candidates assembled onto a carrier are then used to treat separate animals, either by administration, or by ex vivo use, according to the intended purpose. The animals are bled before and after treatment, and the antibody levels in plasma are determined by standard immunoassay as appropriate for the specific antibody. Efficacy of the candidates is then assessed according to the comparative degree in reduction in the antibody level.

In some embodiments, the epitope used lacks T cell epitope(s). Methods for detecting T cell epitopes are well known in the art. For example, various assays which detect T cell proliferation (such as thymidine incorporation) may be used. The presence of T cell epitopes can also be determined by measuring secretion of T cell-derived lymphokines by methods well known in the art. Antigens that fail to induce statistical significant incorporation of thymidine above background (i.e., generally p less than 0.05 using standard statistical methods) are generally considered to lack T cell epitopes, although it will be appreciated that the quantitative amount of thymidine incorporation may vary, depending on the polypeptide being tested. Generally, a stimulation index below about 2-3, more preferably less than about 1, indicates lack of T cell epitopes. Location and content of T cell epitopes are determined empirically.

Removing antibody from biological fluids ex vivo

This invention further includes methods for reducing levels of disease- associated antibodies in the biological fluid of an individual, comprising contacting the fluid with an epitope-presenting carrer ex vivo under conditions that permit the antibodies to bind epitopes on the carrier. Suitable bodily fluids include those that can be returned to the individual, such as blood, plasma, or lymph.

Affinity adsorption apheresis is described generally in Nilsson et al. (1981) Blood 58(1):38-44; Christie et al. (1993) Transfusion 33:234-242; Richter et al. (1997) ASAIO J. 43(1):53-59; Suzuki et al. (1994) Autoimmnumty 19: 105-112; U.S. Pat. No. 5,733,254; Richter et al. (1993) Metabol. Clin. Exp. 42:888-894; Richter et al. (1997) ASAIO J. 43(1):53-59; and Wallukat et al. (1996) Int'l J. Card. 54:191-195.

Accordingly, the invention includes methods of reducing levels of disease-associated antibodies in an individual, comprising treating the individual's blood (including any component thereof which contains antibody) extracoporeally (i.e., outside the body or ex vivo) with an eptiope-presenting carrier under conditions that permit the antibodies to bind the epitope; removing antibody-eptiope-presenting carrier complexes, if any; and returning the blood to the individual.

In the methods of the invention, the bodily fluid is removed from the individual for extracorporeal binding to an epitope-presenting carrier of this invention. For example, apparatuses and methods for removing blood and separating it into its constituent components are known in the art (see, e.g., U.S. Pat. Nos. 4,086,924; 4,223,672). The blood or portions thereof are then exposed to the carrier. The carrier neutralizes (i.e., binds) the unwanted antibody, and the blood components are then returned to the individual.

In a preferred technique, the antibody-carrier complex is removed before the fluid is returned to the individual. This may be done, for example, by using a carrier attached to a solid phase, or by using a soluble carrier and selectively removing the complex from the treated solution.

To create a solid phase, the carrier is adapted to render it insoluble. For example, where the carrier is one of the preferred platforms listed above, then the platform can be chemically adapted during synthesis to include an additional reactive group in the core structure. For example, an additional linkage can be added to a triethlyene glycol structure present in the core. The linkage is then used to attach the platform to an insoluble structure, such as a polystyrene or polyethylene bead, a polycellulose membrane, or other desirable structure. Commercially available matrices include agarose (a neutral linear polysaccharide generally composed of D-galactose and altered 3,6-anhydrogalactose residues, for example Sepharose™, Pharmacia), activated gels, nitrocellulose, borosilicate, glass fiber filters, silica, polyvinylchloride, polystyrene, and diazotized paper. Methods for preparing peptide-peptide conjugates are described in Hermanson, G. T., “Bioconjugate Techniques”, Academic Press: New York, 1996; and in “ Chemistry of Protein Conjugation and Cross-linking” by S. S. Wong, CRC Press, 1993. The biological fluid to be treated is contacted with the solid phase, and antibodies in the fluid complex to the solid phase. The supernatant fluid can then be removed from the solid phase for return to the individual. In some instances, the solid phase can also be cleared of antibody for repeat use by using a suitable wash, providing both the epitope and the carrier is resistant to the washing solution. Suitable washing solutions may include 0.1 M glycine buffer, pH 2.4, dilute acetic acid, or 1 M KSCN buffered to ˜pH 7.

If the carrier is not part of a solid phase, then the antibody-carrier complex can be removed from the fluid by any other appropriate method, including but not limited to microfiltration, antibody capture, or precipitation. Solutions suitable to cause precipitation of the complex depend on the solubility of the complex, and may include ammonium sulfate or polyethylene glycol. If the fluid is to be returned to the individual, then the precipitating solution should be chosen so that any that remains in the fluid does not cause an adverse reaction in the individual.

It is understood that the in vivo and ex vivo methods described herein may used in conjunction with each other.

The invention also contemplates devices which can be used for reducing the level of antibody in a biological fluid using an epitope-presenting carrier of this invention. Typically, the device will be a flow system, comprising the following elements: a) a port that permits biological fluid to flow into the device; b) a chamber in which the fluid is permitted to contact the epitope-bound carrier (optionally in a solid phase); c) a port that permits the treated fluid to flow out of the device. Such devices can be designed as continuous flow systems, and as systems that permit the treatment of a single sample from an individual for purposes of analysis or readministration at a subsequent time.

Administration andformulations Various formulations of epitope-presenting carrier(s) may be used for administration. In some embodiments, the epitope-presenting carrier(s) may be administered neat. Preferably, the epitope-presenting carrier(s) is in a composition comprising an epitope-presenting carrier(s) and a pharmaceutically acceptable excipient, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonperenteral drug delivery are set forth in Remington's Pharmaceutical Sciences 19th Ed. Mack Publishing (1995).

Generally, these compositions are formulated for administration by injection (e.g., intraperitoneally, intravenously, subcutaneously, intramuscularly, etc.). Accordingly, these compositions are preferably combined with pharmaceutically acceptable vehicles such as saline, Ringer's solution, dextrose solution, and the like. Generally, the epitope-presenting carrier will constitute about 0.01% to 10% by weight of the formulation due to practical, empirical considerations such as solubility and osmolarity. The particular dosage regimen, i.e., dose, timing and repetition, will depend on, inter alia, the clinical indication and the particular individual and that individual's medical history. Generally, a dose of about 1 μg to about 100 mg conjugate/kg body weight, preferably about 100 μg to about 10 mg/kg body weight, preferably about 50 μg to about 5 mg/kg body weight, preferably about 1 μg to about 1 g conjugate/kg body weight, preferably about 5 μg to about 500 mg body weight is administered. Empirical considerations, such as the half life, generally will contribute to determination of the dosage. An epitope-presenting carrier may be administered daily, followed by less frequent administrations, such as two times per week, once a week, or even less frequently. In other embodiments, an epitope- presenting carrier(s) is administered less frequently, i.e., bi-weekly, weekly, every ten days, or every two weeks. Frequency of administration may be determined and adjusted over the course of therapy, and is based on maintaining the desired level of antibody. Other appropriate dosing schedules may be as frequent as daily or 3 doses per week, or one dose per week, or one dose every two to four weeks, or one dose on a monthly or less frequent schedule depending on the individual or the disease state. Repetitive administrations may be required to achieve and/or maintain the desired level of antibody. Alternatively, sustained continuous release formulations of the compositions may be appropriate. Various formulations and devices for achieving sustained release are known in the art. Other formulations include those suitable for oral administration, which may be suitable if the epitope-presenting carrier is able to cross the mucosa. Similarly, an aerosol formulation may be suitable.

In some embodiments, more than one epitope-presenting carrier may be present in a composition. Such compositions may contain at least one, at least two, at least three, at least four, at least five different conjugates. Such “cocktails”, as they are often denoted in the art, may be particularly useful in treating a broader range of population of individuals. They may also be useful in being more effective than using only one (or fewer than are contained in the cocktail) epitope-presenting carrier(s).

The compositions may be administered alone or in conjunction with other forms of agents that serve to enhance and/or complement the effectiveness of an epitope-presenting carrier. Additionally, or alternatively, a dosage regimen may begin with one epitope-presenting carrier, and then switch to another.

An individual suitable for administration of an epitope-presenting carrier(s) (or composition comprising an epitope-presenting carrier(s)) is one who exhibits undesirable levels of a disease-associated antibody (such as those described above). Levels of disease-associated antibodies may be determined using standard assays in the art such as ELISA. Preferably, the individual is human. Measurable circulating levels of disease-associated antibody need not be detectable, it may be predictable (due to, for example, risk factors; genetic factors; environmental factors; and other known etiologies) that such antibodies are likely to be produced. Thus, the methods of the invention also pertain to those cases in which a prophylactic effect is contemplated.

In preferred embodiments, the epitope-presenting carrier (such as a valency platform conjugate) is administered such that the duration of the effect is longer than when compared to other epitope-presenting carrier(s). In this vein, considerations such as (a) the particular carrier used; (b) valency; (c) type of epitope); (d) dosage regimen; and (e) means of administration may enter into producing this duration. Any one or more of the above factors may provide a basis for the extent of duration of effect (i.e., length of time that the antibodies are reduced). Similarly, any one or more of the above factors may provide a basis for the rapidity of reduction of antibody.

Kits for use in conjunction with the methods of the invention

The invention also includes kits for use in conjunction with the methods described herein. In some embodiments, the kits effect in vivo reduction of circulating antibody levels (i.e., disease-associated antibody). In other embodiments, the kits effect extracorporeal selective formation and/or removal of immunosorbent- anti-viral antibody complexes (i.e., ex vivo removal). These kits contain components specific for whatever antibody(ies) is targetted for removal. In other embodiments, kits and compositions are provided for use in detection of antibody(ies) to be reduced. These kits aid in assessing (a) whether an individual is indicated for selective removal of disease-associated antibody (for example, if the titer is considered to be above a requisite threshold); (b) monitoring an individual after treatment (i.e., selective removal) to determine whether further sufficient removal has occurred and/or whether further is indicated (for example, when a period of time has elapsed since removal, and the titer of anti-viral antibody has risen to or past a requisite threshold, and treatment with a viral therapeutic agent is still indicated); (c) which antibody(ies) an individual is producing (this would indicate which antibody or antibodies should be selectively removed). These kits contain components specific for antibodies targeted for reduction and/or removal, aiding detection and/or monitoring.

It is understood that these kits, especially those used to effect selective removal of antibody, may also be denoted as “systems”.

Kits of the invention comprise an epitope-presenting carrier (preferably, the carrier is a valency platform molecule) that specifically binds the antibody to be removed in suitable packaging. Preferably, the kit also contains instructions for its use. Appropriate carriers (including those conjugated to epitope) have been discussed above.

The kits of the invention may further comprise reagents for testing for the presence (and/or level) of antibody targetted for reduction and/or removal, which would be useful for monitoring purposes.

The following Examples are provided to illustrate but not limit the present invention.

### EXAMPLES

### Example 1

Synthesis of αGal epitopes

The general analytical methods and characterization techniques used in the present disclosure are identified below. NMR spectra wee recorded on a Bruker AC300 spectrometer at 300 MHz for ¹H and 75 MHz for ¹³C. Chemical shifts were recorded in parts per million (δ) relative to TMS (i.e., tetramethylsilane, δ=0.0 ppm) or to the residual signal of deuterated solvents: chloroform (δ=7.27 ppm for ¹H; δ=77.23 ppm for ¹³C), methanol (δ=4.87 ppm for ¹H; δ=49.15 ppm for ¹³C) and D₂O (δ=4.80 (DSS) ppm for ¹H). Coupling constants (J) are reported in hertz. Analytical HPLC analyses were performed on a Hewlett Packard liquid chromatography HP 1090 instrument fitted with a Vydac C18 column (4.6×250 mm, 5 μm particle size). Preparative HPLC was performed on Dynamax SD 200 system with Vydac C 18 column (22×250 mm, 10 μm particle size). Mass spectra were recorded on Finnigan LCQ mass spectrometer.

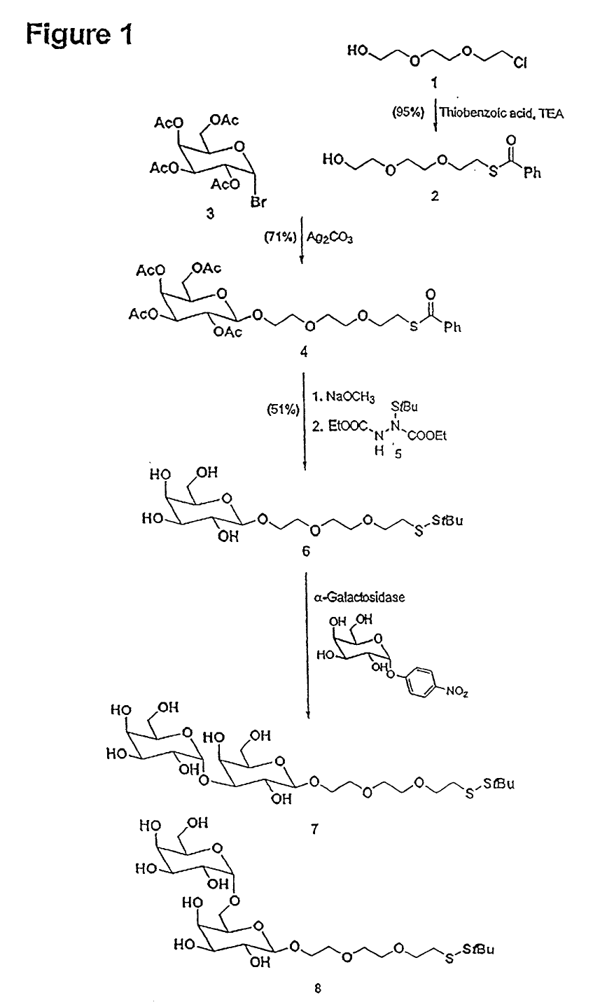

Enzymatic synthesis of the αGal epitope, 2-[2-(2-thioethoxy)ethoxy]ethyl 3-O- (α-D-galactopyranosyl)-β-,galactopyranoside: A reaction scheme illustrating the synthesis is shown in FIG. 1.

### Compound 2

### S-2-[2-(2-Hydroxylethoxy)ethoxy]ethyl thiobenzoate

To a mixture of 2-[2-(2-cloroethoxy)ethoxy]ethanol, compound 1 (20 g, 0.12 mol) and thiobenzoic acid (16.4 g, 0.12 mol) was added 12 g of triethylamine at room temperature. The mixture was then stirred at 90° C. for 1 h. After cooled to room temperature, ethyl acetate (100 mL) was added to the reaction mixture and filtered. The filtrate was concentrated and purified via silica gel chromatography (hexane/ethyl acetate, 1:1) to give compound 2 (30.6 g, 95%) as an orange syrup: ¹H NMR (CDCl₃): δ7.97 (dd, J=8.3, 1.4, 2 H), 7.56 (d, J=7.4, 1 H), 7.45 (t, J=6.9, 2 H), 3.73 (m, 4 H), 3.68 (s, 4 H), 3.62 (m, 2 H), 3.31 (t, 2 H).

### Compound 4

### 2-[2-(2-Benzoylthioethoxy)ethoxy]ethyl 2,3,4,6-tetra-O-acetyl-β-D-galactopyranoside

To a solution of compound 2 (7.79 g, 28.9 mmol) and acetobromo-α-D-galactose, compound 3 (17.80 g, 43.28 mmol) in dry CH₂Cl₂ (100 mL) were added Ag₂CO₃ (9.27 g, 36.1 mmol) and activated 4Å molecular sieve (powder, 10 g) at 0° C. After stirred at room temperature for 3 d, the reaction mixture was filtered through Celite and the filtrate was concentrated and purified via silica gel chromatography (hexane/ethyl acetate, 4:1) to give compound 4 (12.29 g, 71%) as a colorless syrup: ¹H NMR (CDCl₃): δ7.99-7.95 (m, 2 H), 7.61-7.55 (m, 1 H), 7.48-7.43 (m, 2 H), 5.39 (dd, J=1.0, 3.4, 1 H), 5.21 (dd, J=7.9, 10.5, 1 H), 5.03 (dd, J=3.4, 10.5, 1 H), 4.58 (d, J=7.9, 1 H), 4.14 (dd, J=2.9, 6.9, 1 H), 3.99-3.89 (m, 2 H), 3.79-3.65 (m, 10 H), 3.30 (t, J=6.4, 2 H), 2.15-1.98 (4s, 12 H); ¹³C NMR (CDCl₃): δ191.4, 170.3, 170.2, 170.0, 169.4, 136.9, 133.4, 128.5, 127.2, 101.3, 97.4, 70.9, 70.6, 70.5, 70.3, 69.8, 69.0, 68.8, 67.0, 61.2, 20.7, 20.6, 20.5; MS (ESI): m/e (M+Na⁺) Calcd. for C₂₇H₃₆O₁₃SNa: 623.2, obsd.: 623.2.

### Compound 6

### 2-[2-(2-tert-Butyldithioethoxy)ethoxy]ethyl β-D-galactopyranoside

To a stirred solution of compound 4 (3.47 g, 5.77 mmol) in methanol (15 mL) was added NaOCH₃ (0.50 g, 9.28 mmol) at 0° C. After 3 h, diethyl 1-(tert-bytylthio)-1,2-hydrzainedicarboxylate, compound 5 (2.2 g, 8.3 mmol), which was prepared as described (Wunsch, E., et al. Hoppe-Seyler's Z. Physiol. Chem. (1982) 363:1461-1464), was added. The reaction mixture was stirred at room temperature for another 2 h. Dowex 50X2-400 resin was then added to neutralized the solution and filtered. The filtrate was concentrated and purified via silica gel chromatography (CH₂Cl₂/MeOH, 9:1) to give compound 6 (1.22 g, 51%) as a white solid: ¹H NMR (CDCl₃): δ4.39 (br s, 1 H), 4.35 (br d, 2 H), 4.11 (br s, 1 H), 4.02 (br s, 2 H), 3.84 (br s, 2 H), 3.78-3.48 (m, 13 H), 2.89 (t, J=6.7, 2 H), 1.33 (s, 9H); MS (ESI): m/e (M+1) Calcd. for C₁₆H₃₃O₈S₂: 417.2, obsd.: 417.5.

### Compounds 7 and 8

### 2-[2-(2-tert-Butyldithioethoxy)ethoxy]ethyl 3-O-(α-D-galactopyranosyl)-β-D-galactopyranoside(7)

### 2-[2-(2-tert-butyldithioethoxy)ethoxy]ethyl 3-O-(α-D-galactopyranosyl)-β-D-galactopyranoside (8):

To a solution of compound 6 (2.87 g, 6.89 mmol) and ρ-nitrophenyl α-D-galactopyranoside (200 mg) in 10 mL of sodium phosphate buffer (50 mM, pH 6.5) was added 20 mg of coffee bean a-galactosidase. The reaction was proceeded at room temperature with gradual addition of donor until 300 mM ρ-nitrophenol had been formed (3 d). The reaction mixture was lyophilized and the residue was suspended in methanol, filtered and concentrated. The unreacted starting material (2.5 g) was recovered by silica gel chromatography (CH₂Cl₂/MeOH, 95:5 to 70:30). The products were first purified on P2 Gel filtration column eluted with water and then on reverse phase HPLC column to yield compound 7 (80 mg, 2.0%) and compound 8 (106 mg, 2.6%) as white solids. For compound 7: analytical RF-HPLC: t_{R} 8.49 min with a gradient of 25 to 30% ACN in H₂O at a flow rate of 1 mL/min, purity, 100%; ¹H NMR (CD₃OD): 5.04 (d, J=1.9, 1 H), 4.32 (dd, J=1.6, 6.2, 1 H), 4.25 (t, J=5.5, 1 H), 4.05-4.00 (m, 2 H), 3.93 (d, J=1.2, 1 H), 3.83-3.61 (m, 17 H), 3.52 (t, 1 H), 2.89 (t, J=6.6, 2 H), 1.33 (s, 9H); MS (ESI): m/e (M+Na⁺) Calcd. for C₂₂H₄₂O₁₃S₂Na: 601.2, obsd.: 601.2. For compound 8: analytical RF-HPLC: t_{R} 7.14 min with a gradient of 25 to 30% ACN in H₂O at a flow rate of 1 mL/min, purity, 100%; ¹H NMR (CD₃OD): 4.27(d, J=7.5, 1 H), 4.00-3.62 (m, 22 H), 3.50 (m, 1 H), 2.89 (t, J=6.6, 2 H), 1.36 (s, 9 H); MS (ESI): m/e (M+Na⁺) Caled. for C₂₂H₄₂O₁₃S₂Na: 601.2, obsd.: 601.1.

These two disaccharides were fuirther characterized after acetylated with Ac₂O in pyridine at room temperature overnight: for acetylated compound 7: ¹H NMR (CDCl₃): δ5.46 (br d, J=3.3, 1 H), 5.37 (br d, J=3.3, 1 H), 5.31-5.22 (m, 3 H), 5.14 (dd, J=3.3, 10.2, 1 H), 4.50 (d, J=7.8, 1 H), 4.29 (br t, J=6.5, 7.3, 1 H), 4.21 (dd, J=7.3, 11.3, 1 H), 4.14 (d, J=7.3, 2 H), 4.04 (dd, J=6.5, 10.8, 1 H), 3.91 (dd, J=10.4, 2.9, 1 H), 3.84 (br t, J=7.3, 1 H), 3.87-3.62 (m, 10 H), 2.91 (t, J=7.6, 2 H), 2.15-1.95 (7s, 21 H), 1.35 (s, 9 H) and for acetylated compound 8: ¹H NMR (CDCl₃): δ5.42 (br d, J=3.1, 1 H), 5.41 (br d, J=3.0, 1 H), 5.28 (dd, J=3.0, 10.4, 1 H), 5.19 (dd, J=7.8, 10.4, 1 H), 5.12 (dd, J=3.3, 10.4, 1 H), 5.02 (dd, J=3.1, 10.4, 1 H), 4.93 (d, J=3.3, 1 H), 4.57 (d, J=7.8, 1 H), 4.21 (br t, J=6.8, 1 H), 4.09 (m, 2 H), 3.95 (dt, J=3.9, 10.8, 1 H), 3.86 (br t, J=6.6, 1 H), 3.81-3.62 (m, 10 H), 3.44 (dd, J=7.4, 10.2, 1 H), 2.89 (t, J=6.7, 2 H), 2.12-1.98 (7s, 21 H), 1.35 (s, 9 H).

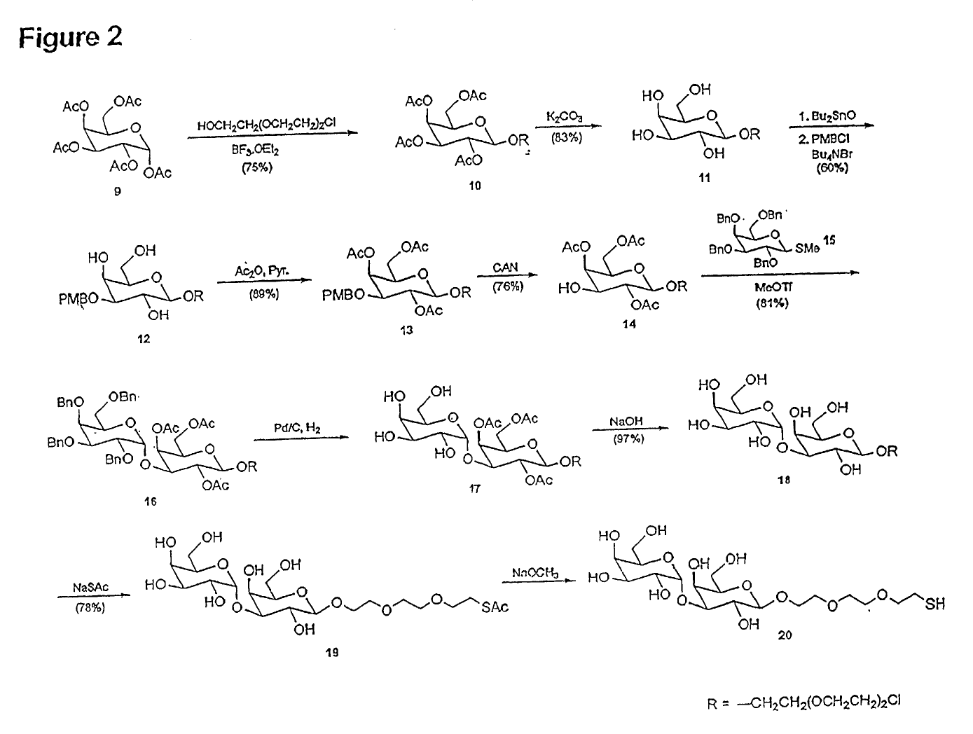

Chemical synthesis of the αGal epitope, 2-[2-(2-thioethoxv)ethoxy]ethyl 3-O-(α-D-galactopyranosyl)-β-D-galactopyranoside: A reaction scheme illustrating this synthesis is shown in FIG. 2.

### Compound 10

### 2-[2-(2-chloroethoxy)ethoxy]ethyl 2,3,4,6-tetra-O-acetyl-β-D-galactopyranoside

To a mixture of galactose pentaacetate, compound 9 (70 g, 179 mmol), 2-[2-(2-chloroethoxy)ethoxy]ethanol (45.4 g, 270 mmol) and activated 4Å molecular sieve (20 g) in dry CH₂Cl₂ (500 mL) was added BF₃Et₂O (52 g, 370 mmol) dropwise at room temperature for 3 h. After stirred for 2 d, the suspension was filtered through Celite and the filtrate was poured into 300 mL of saturated aqueous NaHCO₃ cooled in an ice bath. The organic phase was separated and the aqueous phase was extracted with CH₂Cl₂. The combined organic phases were washed with brine, dried, and concentrated. The residue was purified via silica gel chromatography (hexane/ethyl acetate, 1:1) to give compound 10 (67 g, 75%) as a colorless oil: ¹H NMR (CDCl₃): δ5.36 (d, J=3.2, 1 H), 5.19 (dd, J=10.4, 8.0, 1 H), 5.00 (dd, J=10.4, 3.6, 1 H), 4.55 (d, J=8.0, 1 H), 4.18-4.08 (m, 3 H), 391-3.87 (m, 2 H), 3.74 (m, 2H), 3.72-3.61 (m, 8 H), 2.13 (s, 3 H), 2.04 (s, 3 H), 2.03 (s, 3 H), 1.96 (s, 3 H).

### Compound 11

### 2-[2-(2-chloroethoxy)ethoxy]ethyl β-D-galactopyranoside

A solution of compound 10 (67 g, 134 mmol) in 200 mL of methanol and 250 mL of 1 M K₂CO₃ aqueous solution was stirred at room temperature overnight. The reaction mixture was poured into 700 mL of methanol cooled with ice-water bath. The precipitate was filtered through celite and washed with methanol. The filtrate was combined and neutralized with Dowex resin (H form) until pH 6. The resin was filtered and washed with water. The filtrate was concentrated and lyophilized to give compound 11 (37 g, 83%) as a colorless oil: ¹H NMR (D₂O): δ4.30 (d, J=7.2, 1 H), 3.95 (m, 1 H), 3.80 (d, J=3.6, 1 H), 3.80-3.50 (m, 15 H), 3.40 (m, 1 H).

### Compound 12

### 2-[2-(2-chloroethoxy)ethoxy]ethyl 3-O-ρ-methoxybenzyl-β-D-galactopyranoside

A mixture of compound 11 (37 g, 112 mmol) and dibutyltin oxide (46 g, 210 mmol) in dry MeOH (300 mL) was refluxed under nitrogen until clear (10 hr). The reaction mixture was concentrated and the residue was dried under vacuum. The residue was dissolved in 800 mL of dioxane and 80 mL of DMF and ρ-methoxybenzyl chloride (32 g, 28 ml, 0.20 mol) was added. The resulting mixture was stirred at 100° C. for 10 h to give a brownish solution with precipitate. After cooled to room temperature, the precipitate was removed by filtration through Celite and washed with dioxane (100 mL) and chloroform (100 mL). The combined organic phases were concentrated and purified by silica gel chromatography (ethyl acetate) to give compound 12 (30 g, 60%) as a colorless oil.

### Compound 13

### 2-[2-(2-chloroethoxy)ethoxy]ethyl 2,4,6-tri-O-acetyl-3-O-ρ-methoxybenzyl β-D-galactopyranoside

Compound 12 (30 g, 66.5 mmol) was acetylated with Ac₂O (150 mL) in pyridine (150 mL), catalyzed by DMAP (120 mg). After stirred for 5 h, the reaction mixture was concentrated. The residue was dissolved in chloroform (300 mL) and washed with HCI solution (0.5 M), water, saturated aqueous NaHCO₃ and brine. The organic phase was dried over anhydrous Na₂SO₄ and concentrated to give compound 13 (34 g, 89%) as a colorless oil: ¹H NMR (CDCI₃): δ7.19 (d, J=8.6, 2 H), 6.87 (d, J=8.6,2 H), 5.48 (d, J=3.3, 1 H), 5.10 (dd, J=10.0, 8.1, 1 H),4.62 (d, J=11.6, 1 H), 4.45 (d, J=8.1, 1 H), 4.34 (d, J=11.6, 1 H), 4.17 (dd, J=6.7, 1.0, 2 H), 3.94 (m, 1 H), 3.82-3.61 (m, 15 H), 3.49 (dd, J=10.0, 3.3, 1 H), 2.16 (s, 3 H), 2.08 (s, 3 H), 2.04 (s, 3 H); MS (ESI): m/e (M+Na⁺) Caled. for C₂₆H₃₇ClO₁₂Na: 599.2, obsd: 599.4.

### Compound 14

### 2-[2-(2-chloroethoxy)ethoxy]ethyl 2,4,6-tri-O-acetyl β-D-galactopyranoside

To a solution of compound 13 (34 g, 59 mmol) in 300 mL of CH₃CN/water (9:1) was added CAN (64 g, 120 mmol) slowly during 3 h at 0° C. After addition, the mixture was stirred at the same temperature for 3 h. The reaction mixture was then concentrated, diluted with 250 mL of water, and extracted with chloroform. The organic extracts were washed with saturated aqueous NaHCO₃ and brine, dried over anhydrous Na₂SO₄, and concentrated. The residue was purified via flash column chromatography (hexane/ethyl acetate, 3:1 to 2:3) to give compound 14 (20.4 g, 76%) as a colorless oil: ¹H NMR (CDCl₃): δ5.31 (d, J=3.6, 1 H), 4.95 (dd, J=10.0, 7.8, 1 H), 4.51 (d, J=8.0, 1 H), 4.13 (m, 2 H), 3.94 (m, 1 H), 4.00-3.61 (m, 14 H), 2.15 (s, 3 H), 2.11 (s, 3 H), 2.04 (s,3 H).

### Compound 16

### 2-[2-(2-chloroethoxy)ethoxy]ethyl 3-O-(2,3,4,6-tetra-O-benzyl-α-D-galactopyranosyl)-2,4,6-tri-O-acetyl-β-D-galactopyranoside

To a mixture of compound 14 (10 g, 22 mmol), compound 15 (25.4 g, 45 mmol), 4-methyl-2,6-di-t-butyl pyridine (6.8 g, 33 mmol), and activated 4Å molecular sieve (10 g) in dry ether (300 mL) was added a solution of methyl triflate (7.2 mL) in ether (50 mL) dropwise via a syringe-pump over a period of 24 h. After stirred at room temperature for 36 h, the reaction mixture was filtered through Celite. The filtrate was concentrated and purified via silica gel chromatography (hexane/ethyl acetate, 4:1 to 1:1) to give compound 16 (17.3 g, 81%) as a yellowish oil: ¹H NMR (CDCl₃): δ7.35-7.22 (m, 20 H), 5.43 (d, J=3.2, 1 H), 5.16 (dd, J=10.0, 8.0, 1 H), 5.06 (d, J=3.4, 1 H), 4.90 (d, J=11.6, 1 H), 4.81 (d, J=12.0, 1 H), 4.68-4.62 (m, 3 H), 4.46-4.39 (m, 4 H), 4.12-3.59 (m, 20 H), 3.49 (d, J=6.4, 2 H), 2.03 (s, 3 H), 1.94 (s, 3 H), 1.79(s, 3 H); MS (ESI): m/e (M+Na⁺) Calcd. for C₅₂H₆₃ClO₁₆Na: 1001.4, obsd: 1001.8.

### Compound 17

### 2-[2-(2-chloroethoxy)ethoxy]ethyl 3-O-α-D-galactopyranosyl-2,4,6-tri-O-acetyl-β-D-galactopyranoside

A mixture of compound 16 (18.1 g, 18.5 mmol) and Pd/C (20%, 3 g) in 300 mL of methanol and 1.5 mL of acetic acid was shaken under compressed hydrogen (50 psi) at room temperature for 9 h. The reaction mixture was filtered through Celite and concentrated. Water (10 mL) was added to the residue to assist the removal of acetic acid and the crude product was used directly in next reaction without purification. ¹H NMR (CD₃OD): δ5.52 (dd, J=10.0, 8.4, 1 H), 4.98 (d, J=3.6, 1 H), 4.62 (d, J=8.0, 1 H), 4.14 (m, 2 H), 4.06 (m, 1 H), 3.98 (t, J=8.4, 1 H), 3.92 (m, 1 H), 3.86 (d, J=2.7, 1 H), 3.77-3.61 (m, 16 H), 3.55 (dd, J=10.0, 3.2, 1 H), 2.13 (s, 3 H), 2.11 (s, 3 H), 2.03 (s, 3 H). MS (ESI): m/e (M+Na⁺) Calcd. for C₂₄H₃₉ClO₁₆Na: 641.2, obsd: 641.3.

### Compound 18

### 2-[2-(2-chloroethoxy)ethoxy]ethyl 3-O-α-D-galactopyranosyl-β-D-galactopyranoside

A solution of compound 17 in 40 mL of IM NaOH and 300 mL of methanol was stirred at room temperature for 6 h. The reaction mixture was neutralized with Dowex resin (H form) to pH 6, filtered and lyophilized to give compound 18 (8.9 g, 97%) as a white solid. ¹H NMR (D₂O): δ5.18 (d, J=3.7, 1 H), 4.53 (d, J=7.7, 1 H), 4.25-3.68 (m, 24 H). MS (ESI): m/e (M+Na⁺) Calcd. for C₁₈H₃₃ClO₁₃Na: 515.2, obsd: 515.5.

### Compound 19

### 2-[2-(2-acetylthioethoxy)ethoxy]ethyl 3-O-(α-D-galactopyranosyl)-β-D-galactopyranoside

A solution of compound 18 (8.9 g, 18.1 mmol) in 100 mL of 1 M potansium thioacetate was stirred at 95° C. under nitrogen for 36 h. The reaction mixture was cooled and loaded on a column packed with Dowex ion exchange resin (H form, 100 g) and eluted with water. The aqueous solution was neutralized with Dowex Marathon WBA anion exchange resin (from pH 2 to 5), filtered, and lyophilized to give compound 19 (7.5 g, 78%) as an off-white solid. ¹H NMR (D₂O): δ5.17 (d, J=3.8, 1 H), 4.52 (d, J=7.7 Hz, 1 H), 4.24-3.67 (m, 25 H), 3.15 (t, J=6.2, 2 H); MS (ESI): m/e (M+Na⁺) Calcd. for C₂₀H₃₆O₁₄SNa: 555.2, obsd: 555.3.

### Compound 20

### 2-[2-(2-thioethoxy)ethoxy]ethyl 3-O-(α-D-galactopyranosyl)-β-D-galactopyranoside

A mixture of compound 19 (650, 1.22 mmol) and 4 g of DOWEX 550A OH anion-exchange resin, pre-washed with methanol, in 50 mL of methanol was stirred at room temperature overnight. The reaction mixture was filtered and the resin was washed with 5% acetic acid in methanol. The filtrate was concentrated to give compound 20 (578 mg, 97%) as a white solid: ¹H NMR (CD₃OD): δ5.00 (d, J=3.6, 1 H), 4.35 (d, J=7.6 Hz, 1 H), 4.04 (m, 2 H), 3.93 (m, 1 H), 3.84 (m, 1 H), 3.82 (dd, J=10.4, 3.2, 1 H), 3.73-3.51 (m, 18 H), 2.83 (t, J=6.0, 2 H); ¹³C NMR (D₂O): δ103.2, 95.8, 77.8, 75.4, 72.7, 71.4, 70.2, 70.1, 69.9, 69.7, 69.2, 68.9, 68.7, 65.4, 61.5, 37.9, 23.5; MS (ESI): m/e (M+Na⁺) Calcd. for C₁₈H₃₄O₁₃SNa: 513.5, obsd: 513.3.

The synthetically prepared αGal epitope on resin was antigenically active as demonstrated by its ability to remove >95% of anti-αGal Ig from normal rhesus monkey or human serum, as measured by FACS.

### Compound 22

### ρ-aminophenyl 3-O-α-D-galactopyranosyl-x-D-galactopyranoside

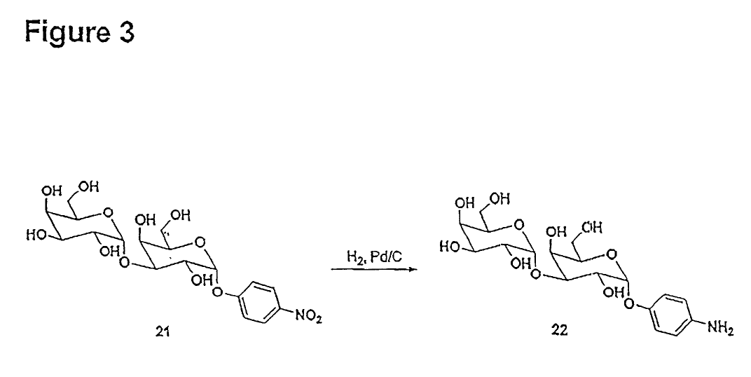

A reaction scheme illustrating the chemoenzymatic synthesis of the αGal epitope, ρ-aminophenyl 3-O-α-D-galactopyranosyl-α-D-galactopyranoside (22) is shown in FIG. 3. ρ-Nitrophenyl 3-O-α-D-galactopyranosyl-α-D-galactopyranoside (compound 21) was prepared enzymatically as described by Nilsson (Tetrahedron Lett. (1997) 38:133-136). A mixture of ρ-nitrophenyl 3-O-α-galactopyranosyl-α-D-galactopyranoside (70 mg, 0.15 mmol) and 10 mg Pd/C (10%) in methanol (4 mL) was stirred under hydrogen at room temperature overnight. The reaction mixture was then filtered through Celite and the filtrate was concentrated in vacuo to give (50 mg) as a yellowish solid: ¹H NMR (CD₃OD): β6.87 (t, 2 H), 6.57 (d, 2 H), 5.21 (d, 1 H), 4.98 (d, 1 H), 4.2-4.1 (m, 2 H), 4.1-3.8 (m, 3 H), 3.8-3.7 (m, 3 H), 3.7-3.5 (m, 4 H); ¹³C NMR (CD₃OD): δ143.8, 120.1, 117.9, 115.4, 101.2, 97.6, 78.3, 77.0, 72.6, 71.5, 71.4, 70.3, 68.7, 67.4, 63.2, 62.6; MS (ESI): m/e (M+Na⁺) Calcd. for C₁₈H₂₇NO₁₁Na: 456.2, obsd: 456.2.

### Example 2

Synthesis of valency platforms

### Synthesis of compound 23

A solution of 1,4-diaminobutane and NaHCO₃ in water/dioxane 1/1 is treated with bromoacetic anhydride. The mixture is extracted with CH₂Cl₂, and the CH₂Cl₂ layer is dried and concentrated to give crude product which is purified by silica gel chromatography to give compound 23.

### Synthesis of compound 24

A solution of 4,7,10-trioxa-1,3-tridecanediamine and NaHCO₃ in water/dioxane 1/1 is treated with bromoacetic anhydride. The mixture is extracted with CH₂Cl₂, and the CH₂Cl₂ layer is dried and concentrated to give crude product which is purified by silica gel chromatography to give compound 24.

### Synthesis of Compound 29

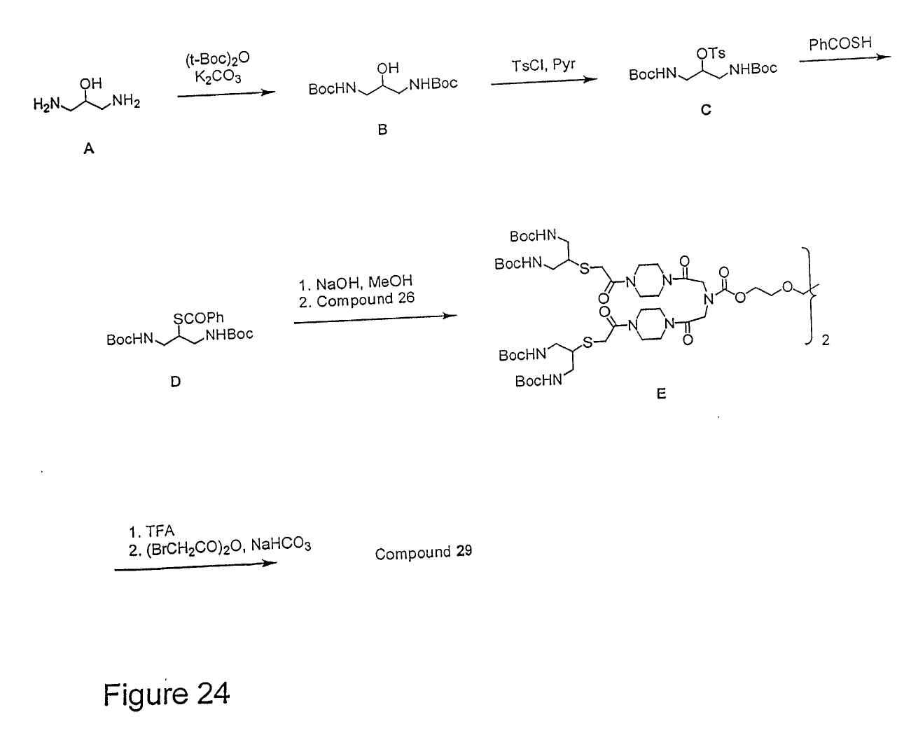

A strategy for synthesis of compound 29 is shown in FIG. 24.

Compound A: A solution of 1,3-diamino-2-hydroxypropane in aqueous dioxane was treated with di-t-butyldicarbonate and Na₂CO₃. The mixture was extracted with CH₂Cl₂, and the CH₂Cl₂ layer was dried and concentrated to give crude product which was purified by silica gel chromatography to give compound B.

Compound B was treated with ρ-toluensulfonyl chloride in pyridine. The mixture was acidified with aqueous HCl and extracted with CH₂Cl₂. The CH₂Cl₂ layer was dried and concentrated to give crude product which was purified by silica gel chromatography to give compound C.

A solution of compound C in a suitable solvent is treated with thiobenzoic acid and a suitable base. The mixture is extracted with CH₂Cl₂, and the CH₂Cl₂ layer is dried and concentrated to give crude product which is purified by silica gel chromatography to give compound D.

A solution of compound D in MeOH is treated with one equivalent of NaOH until the thiobenzoate ester is hydrolyzed as evidenced by TLC. To the resulting mixture is added 0.25 equivalents of compound 26. The mixture is stirred until complete as evidenced by TLC. The mixture is acidified with aqueous H₂SO₄ and extracted with CH₂Cl₂. The CH₂Cl₂ layer is dried and concentrated to give crude product which is purified by silica gel chromatography to give compound E.

Compound E is treated with trifluoroacetic acid to remove the BOC protecting groups. The mixture is concentrated, and the residue is dissolved in a solution of NaHCO₃ in 1/1 dioxane/water. To the resulting solution is added eight equivalents of bromoacetic anhydride. The mixture is stirred until complete as evidenced by TLC. The mixture is acidified with aqueous H₂SO₄ and extracted with CH₂Cl₂. The CH₂Cl₂ layer is dried and concentrated to give crude product which is purified by silica gel chromatography to give compound 29.

### Synthesis of Compound 30

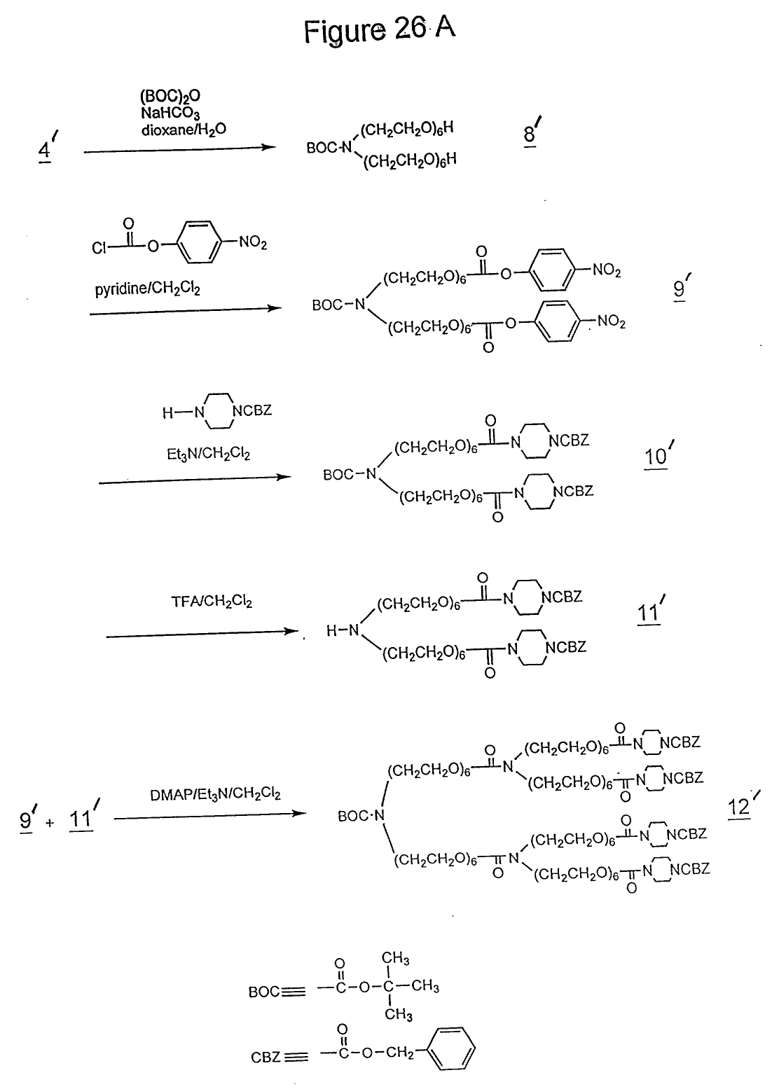

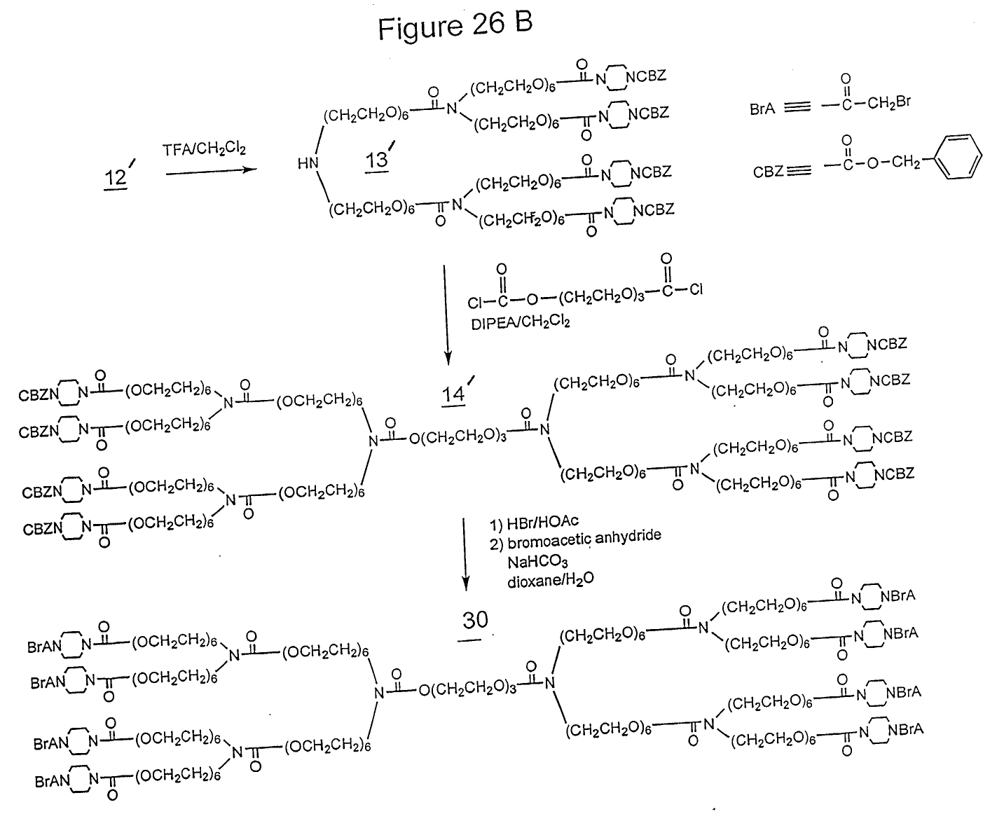

A chemical scheme for the preparation of an octamer of HEGA/TEG is shown in FIGS. 26A and 26B. Compound 30. The bis-hexaethyleneglycolamine (compound +E,uns 4′) was reacted with di-tert-butyldicarbonate to yield the N-BOC compound (compound +E,uns 8′), which was then reacted withpara- nitrophenylchloroformate to yield the para-nitrophenylcarbonate compound (compound +E,uns 9′). The para-nitrophenylcarbonate (PNP) group was then converted to a carbamate group by reaction with mono-CBZ-protected piperazine, yielding compound +E,uns 10′. The BOC group was removed using trifluoroacetic acid to yield compound +E,uns 11′. Compounds +E,uns 9′ and +E,uns 11′ were then reacted together to form a “one-sided” dendritic compound (compound +E,uns 12′). Again, the BOC group was removed using trifluoroacetic acid to yield compound +E,uns 13′. Compound 13′ was then reacted with triethyleneglycol bis chloroformate (from which the “core” is derived) to yield the “two-sided” dendritic compound (compound +E,uns 14′). The terminal CBZ-protected amino groups were then converted to the hydrobromide salt of amino group, and fuirther reacted with bromoacetic anhydride to yield reactive bromoacetyl groups at each of the termini in compound 30.

### Synthesis of Compounds 31 and 32

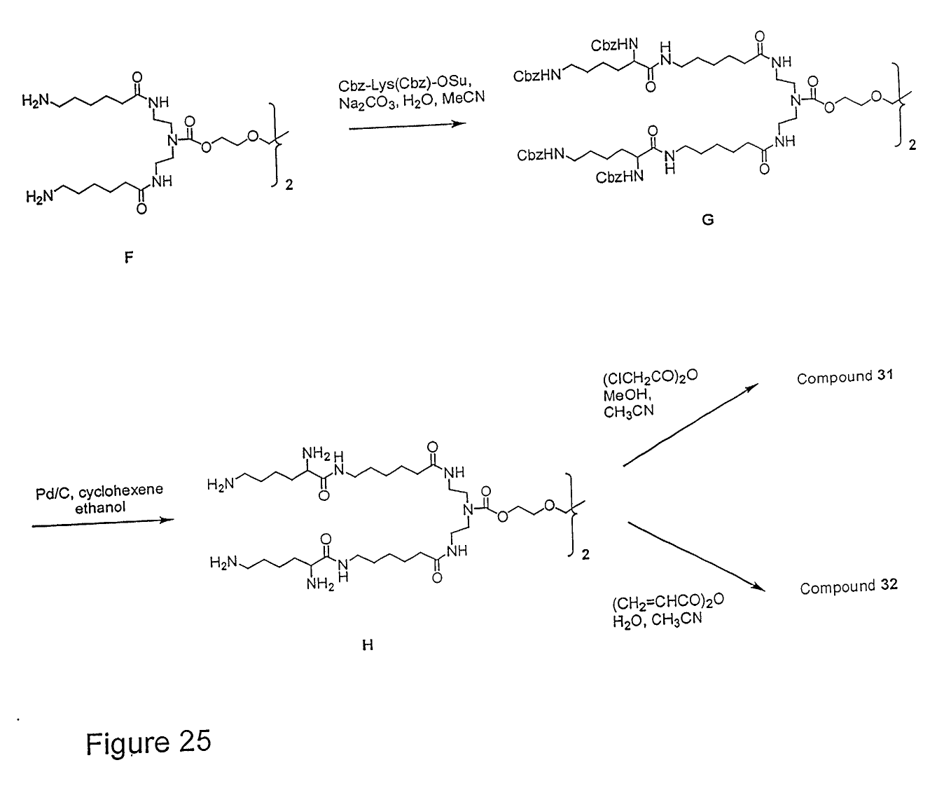

A strategy for synthesis of compound 31 and 32 is shown in FIG. 25.

Tetraamino platform, compound F, was reacted with the N-hydroxysuccinimidyl ester of N_{α},N_{ε}-bis-CBZ-lysine in a solution of water/acetonitrile which contained Na₂CO₃. The acetonitrile was removed under vacuum, and the product precipitated. The precipitate was washed with water and recrystallized from acetonitrile to give G.

The CBZ groups were removed from compound G by catalytic hydrogenation using 10% Pd on carbon in ethanol. The mixture was filtered, and the filtrate was concentrated to give the octa-amine, compound H, as a brown oil.

Compound H was reacted with chloroaceticanhydride in methanol/acetonitrile at room temperature overnight. The crude product was purified by silica gel chromatography to give compound 31.

### Compound 32

A solution of compound H is reacted with acryloyl chloride or acrylic anhydride in the presence of suitable base. The solvent is removed, and the crude product is purified by silica gel chromatography to give compound 32.

### Example 3

Synthesis of αGal conjugates

### Compound 33

### Synthesis of monomeric uGal Conjugate

A mixture of αGal 20 (100 mg, 0.204 mmol), chloroacetamide (38 mg, 0.408 mmol), and tributylphosphine (10 μL) in 1 mL of Na₂CO₃ (10 mg/mL) solution in water/ACN (1:1) was stirred at room temperature overnight. After removing the organic solvent, the remaining aqueous solution was purified on reversed phase HPLC column eluted at 10 mL/min with a gradient of acetonitrile-water (5 to 15%) over 40 minutes to give 33 (96.3 mg, 86%) as a white solid: analytical RF-HPLC: t_{R} 4.78 min with a gradient of 5 to 20% ACN in H₂O at a flow rate of 1 mL/min. purity, 100%; MS (ESI): m/e (M+Na⁺) Calcd. for C₂₀H₃₇NO₁₄SNa: 570.2, obsd: 570.3.

### Compound 34

### Synthesis of dimeric αGal Conjugate

A mixture of αGal 20 (65 mg, 0.133 mmol) in 1 mL of Na₂CO₃ (20 mg/mL) aqueous solution was stirred at room temperature overnight. The solution was purified on reversed phase HPLC column eluted at 10 mL/min with a gradient of acetonitrile-water (10 to 25%) over 40 minutes to give 34 (31.3 mg, 48%) as a white solid: analytical RF-HPLC: t_{R} 9.96 min with a gradient of 5 to 30% ACN in H₂O at a flow rate of 1 mL/min. purity, 100%; MS (ESI): m/e (M+Na⁺) Calcd. for C₃₆H₆₆NO₂₆S₂Na: 1001.3, obsd: 1001.3.

### Compound 35

### Synthesis of Dimeric αGal Conjugate

A mixture of αGal 19 (30 mg, 0.056 mmol), dimeric platform 23 (9.3 mg, 0.028 mmol), and tributylphosphine (0.030 mL) in 3 mL of Na₂CO₃ solution (20 mg/mL) and 2 mL of ACN was stirred under N₂ at room temperature overnight. After removing the organic solvent, the remaining aqueous solution was purified on reversed phase HPLC column eluted at 10 mL/min with a gradient of acetonitrile-water (5 to 15%) over 40 minutes to give 35 (10 mg, 31%) as a white solid: analytical RF-HPLC: t_{R} 8.69 min with a gradient of 5 to 30% ACN in H₂O at a flow rate of 1 mL/min. purity, 97.9%; ¹H NMR (D₂O): δ5.09 (d, J=3.5, 2 H), 4.39 (d, J=7.3, 2 H), 4.21 (t, J=6.5, 2 H), 4.10 (d, J=2.5, 2 H), 4.05 (m, 2 H), 3.96 (d, J=2.6, 2 H), 3.88 (d, J=2.5, 2 H), 3.85 (d, J=3.0, 2 H), 3.82-3.57 (m, 32 H), 3.27 (s, 4 H), 3.23 (s, 4 H), 2.79 (t, J=6.5, 4 H), 1.56 (br s, 4 H); ¹³ C NMR (D₂O): δ173.1, 105.6, 103.4, 98.2, 96.0, 80.2, 78.3, 77.2, 75.3, 73.16, 71.8, 71.3, 70.9, 69.8, 69.4, 69.1, 65.4, 32.4, 40.5, 36.5, 34.6, 32.8; MS (ESI): m/e (M+Na⁺) Calcd. for C₄₄H₈₀N₂O₂₈S₂Na: 1171.4, obsd: 1172.5.

### Compound 36

### Synthesis of Dimeric αGal Conjugate

This compound was prepared following the procedure described above for compound 35. Compound 19 (30 mg, 0.056 mmol) was conjugated with dimeric platform 24 (13.0 mg, 0.028 mmol) to give 36 as a white solid: analytical RF-HPLC: t_{R} 11.3 min with a gradient of 5 to 30% ACN in H₂O at a flow rate of 1 mL/min. purity, 97.4%; ¹H NMR (D₂O): δ5.19 (d, J=3.5, 2 H), 4.53 (d, J=8.8, 2 H), 4.22 (t, J=3.9, 4 H), 4.13 (m, 2 H), 4.05-3.98 (m, 2 H), 3.91 (d, J=2.7, 2 H), 3.88-3.66 (m, 42 H), 3.63 (t, J=6.5, 4 H), 3.35 (t, J=7.2, 8 H), 2.84 (t, J=6.4, 4 H), 1.81 (m, 4 H); MS (ESI): m/e (M+Na⁺) Calcd. for C₅₀H₉₂N₂O₃₁S₂Na: 1303.5, obsd: 1303.4.

### Compound 37

### Synthesis of Tetrameric (Gal Conjugate

The tert-butylthio protecting group of αGal 7 (30 mg, 0.052 mmol) was removed by reducing with tributylphosphine (25 ,μL) in 5 mL of water at room temperature overnight. The reaction mixture was concentrated and dried under high vacuum overnight to remove any residual tert-butylthiol. The residue was dissolved in 5 mL of Na₂CO₃ (10 mg/mL) solution in water/ACN (1:1). Tetrameric platform 25 (5.0 mg, 0.0074 mmol) was added and the resulted solution was stirred at room temperature overnight. After removing the organic solvent, the remaining aqueous solution was purified on a reverse phase HPLC column eluted at 10 mL/min with a gradient of acetonitrile-water (20 to 25%) over 40 minutes to give 37 (9.6 mg, 56%) as a white solid after lyophilization: MS (ESI): m/e (M/2+Na⁺) Calcd. for C₄₇H₇₉O₂₈S₂Na: 1178.4, obsd.: 1178.4.

### Compound 38

### Synthesis of Tetrameric αGal Conjugate

This compound was prepared following the procedure described above for compound 37. Compound 7 (22 mg, 0.038 mmol) was conjugated with platform 26 (7.0 mg, 0.0057 mmol). The product was purified on a reverse phase HPLC column with a gradient of acetonitrile-water (15 to 20%) over 40 minutes to yield 38 (13 mg, 80%) as a white solid: analytical RF-HPLC: t_{R} 5.24 min with a gradient of 15 to 20% ACN in H₂O at a flow rate of 1 mL/niin, purity, 100%; MS (ESI): m/e (M/2+Na⁺) Calcd. for C₅₆H₉₆N₅O₃₃S₂Na: 1454.6, obsd.: 1454.5.

### Compound 39

### Synthesis of Tetrameric αGal Conjugate

This compound was prepared following the procedure described above for compound 38. The conjugation of compound 7 (22 mg, 0.038 mmol) with platform 27 (6.0 mg, 0.0048 minol) yielded 39 (12 mg, 93%) as a white solid: analytical RF-HPLC: t_{R} 11.32 min with a gradient of 15 to 20% ACN in H₂O at a flow rate of 1 mL/min, purity, 100%; MS (ESI): m/e (M/2 +Na⁺) Calcd. for C₅₃H₈₇N₄O₃₂S₂Na: 1379.1, obsd.: 1379.1.

### Compound 40

### Synthesis of Tetrameric αGal Conjugate

This conjugate is prepared by following the procedure described above for compound 38 using αGal 7 and platform 28.

### Compound 41

### Synthesis of Tetrameric αGal Conjugate 41

A mixture of ρ-aminophenyl 3-O-α-D-galactopyranosyl-a-D-galactopyranoside 22 (11 mg, 0.025 mmol), tetrameric platform 28, and NaHCO₃ (3 mg, 0.030 mmol) in 0.15 mL of H₂O/CH₃CN (1:1) was slightly shaken for 1 h. After addition of 0.15 mL of H₂O, the reaction mixture was set at room temperature for 2 d and purified on reversed phase HPLC column eluted at 1 mL/min with a gradient of acetonitrile-water (0 to 30%) over 15 minutes to yiled 41(4.6 mg, 40%) as a white solid: MS (ESI): m/e (M/2+1) Calcd. for C₆₀H₉₅N₇O₂₉: 1377.6 obsd: 1377.9.

### Compound 42

### Synthesis of Tetrameric αGal Conjugate

A solution of the beta isomer of cαGal 7, 2-[2-(2-thioethoxy)ethoxy]ethyl 3-O- (β-D-galactopyranosyl)-β-D-galactopyranoside (30 mg, 0.061 mmol), platform 25 (5 mg, 0.0074 mmol), and tributylphosphine (0.10 mL) in 5 mL of Na₂CO₃ (10 mg/mL) solution in water/ACN (1:1) was stirred at room temperature overnight. After removing the organic solvent, the remaining aqueous solution was purified on a reverse phase HPLC column eluted at 10 mL/min with a gradient of acetonitrile- water (20 to 25%) over 40 minutes to give 42 (2.4 mg, 14%) as a white solid: MS (ESI): m/e (M/2+Na⁺) Calcd. for C₄₇H₇₉O₂₈S₂Na: 1178.4, obsd.: 1179.0.

### Compound 43

### Synthesis of Tetrameric oαGal Conjugate

This conjugate was prepared by following the procedure described above for compound 38. The conjugation of compound 8 (25 mg, 0.043 mmol) with platform 27 (6.0 mg, 0.0048 mmol) yielded 43 (8.8 mg, 68%) as a white solid: analytical RF-HPLC: t_{R} 6.84 min with a gradient of 15 to 20% ACN in H₂O at a flow rate of 1 mL/min, purity, 100%; MS (ESI): m/e (M/2+Na⁺) Calcd. for C₅₃H₈₇N₄O₃₂S₂Na: 1379.4, obsd.: 1379.1.

### Compound 44

### Synthesis of Octameric αGal Conjugate

A solution of αGal 19 (23 mg, 0.429 mmol) and platform 29 (10 mg, 0.0043 mmol) in 2 mL of Na₂CO₃ (10 mg/mL) solution in water/ACN (1:1) was stirred under N₂ at room temperature overnight. The reaction mixture was concentrated and purified on a reverse phase HPLC column eluted at 10 mL/min with a gradient of acetonitrile-water (10 to 30%) over 40 minutes to give 44 (8.5 mg, 37%) as a white solid.

### Compound 45

### Synthesis of Octameric αGal Conjugate

This compound was prepared following the procedure described above for compound 44. Compound 19 (50 mg, 0.094 mmol) was conjugated with platform 30 (10 mg, 0.0018 mmol). The product was purified on a reverse phase HPLC column with a gradient of acetonitrile-water (10 to 50%) over 40 minutes to yield 45 (10.8 mg, 67%) as a white solid: analytical RF-HPLC: t_{R} 11.16 min with a gradient of 5 to 70% ACN in H₂o at a flow rate of 1 mL/min, purity, 100%.

### Compound 46

### Synthesis of Octameric αGal Conjugate

This compound was prepared following the procedure described above for compound 44. Compound 19 (691 mg, 1.41 mmol) was conjugated with platform 31 (280 mg, 0.141 mmol). The product was purified on a reverse phase HPLC column with acetonitrile-water (19.5%) over 40 minutes to yield 46 (604 mg, 76%) as a white solid: analytical RF-HPLC: t_{R} 9.16 min with a gradient of 15-25% ACN in H₂O at a flow rate of 1 mL/min, purity, 100%; ¹H NMR (D₂O): δ5 5.13 (d, J=3.8, 8 H), 4.47 (d, J=7.8, 8 H), 4.12-4.16 (m, 24 H), 4.12-4.05 (m, 8 H), 4.00 (d, J=0.8, 8 H), 3.99-3.92 (m, 8 H), 3.86-3.64 (m, 144 H), 3.39-3.34 (m, 24 H), 3.28 (S, 8 H), 3.22-3.11 (m, 16 H), 2.79 (dd, J=6.1, 10.6, 16 H), 2.20 (t, J=7.1, 8 H), 1.82-1.68 (m, 8 H), 1.59-1.20 (m,40 H); ¹³C NMR (D₂O): δ178.5, 178.4, 175.1, 174.2, 174.0, 159.3, 104.4, 97.0, 78.9, 76.6, 72.5, 71.4, 71.3, 71.1, 71.0, 70.9, 70.8, 70.3, 69.9, 66.5, 62.6, 55.9, 48.5, 48.1, 41.1, 40.8, 39.1, 38.9, 37.4, 37.0, 36.5, 33.0, 32.9, 32.6, 29.8, 29.6, 27.4, 26.7, 24.3; MS (ESI): m/e (M/3 +Na⁺) Calcd. for (C₂₂₄H₄₀₀N₁₈O₁₂₆S₈)/3+Na: 1895.7, obsd.: 1895.3.

### Compound 47

### Synthesis of Octameric (αGal Conjugate

This compound was prepared following the procedure described above for compound 44. Compound 19 (29 mg, 0.055 mmol) was conjugated with platform 32 (10 mg, 0.0055 mmol). The product was purified on reverse phase HPLC column with a gradient of acetonitrile-water (15 to 20%) over 40 minutes to yield 47 (20 mg, 65%) as a white solid: analytical RF-HPLC: t_{R} 11.35 min with a gradient of 15 to 25% ACN in H₂O at a flow rate of 1 mL/min. purity, 82%; MS (ESI): m/e (M/3+Na⁺) Calcd. for (C₂₃₂H₄₁₆N₁₈O₁₂₆S₈)/3+Na: 1933.1, obsd.: 1932.1.

### Example 4

In vitro characterization of αGal conjugates

Materials and Methods

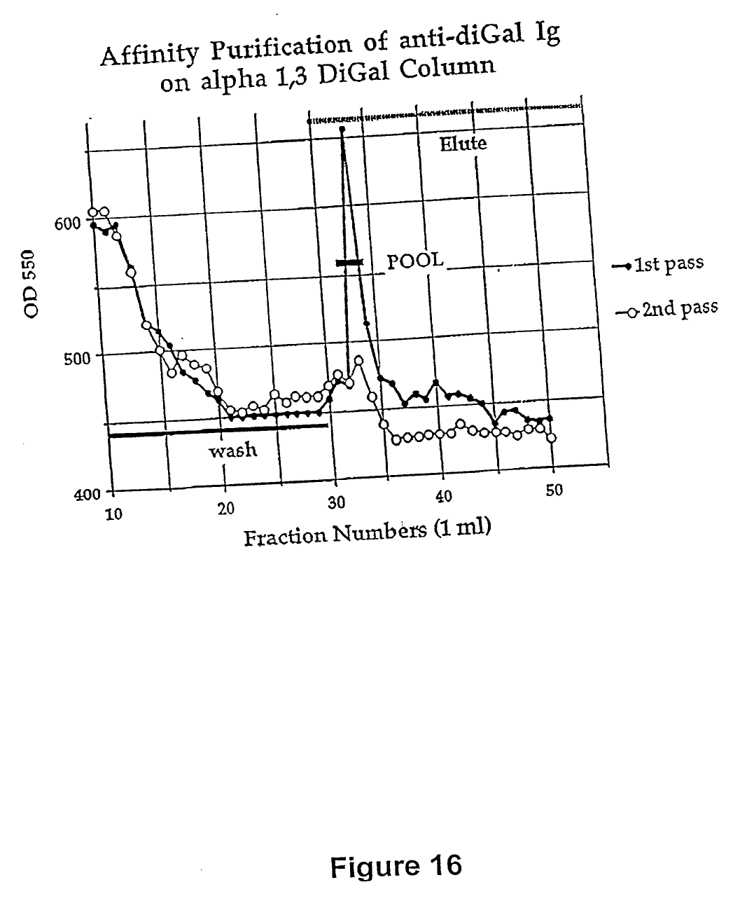

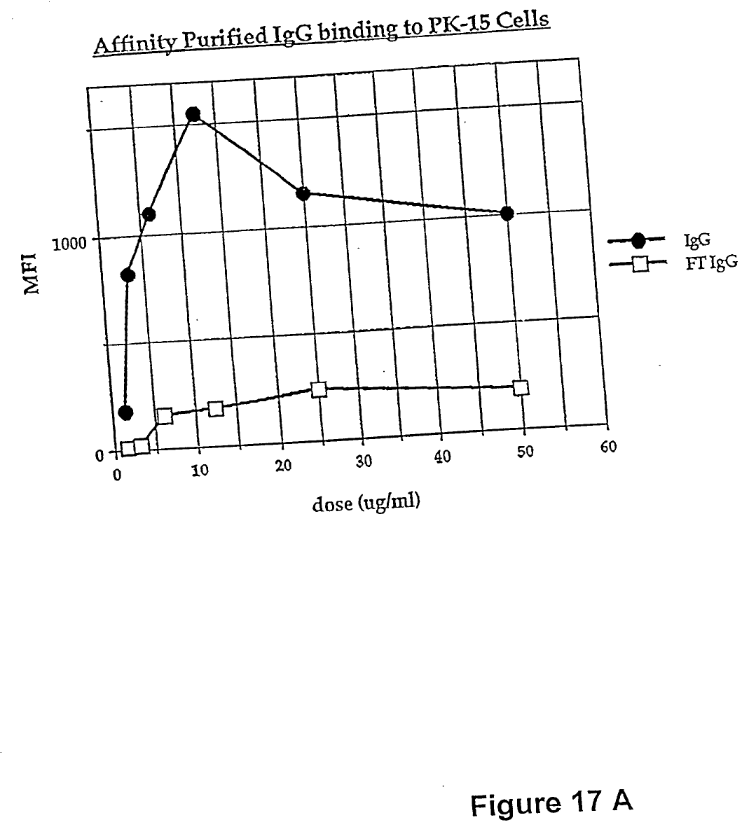

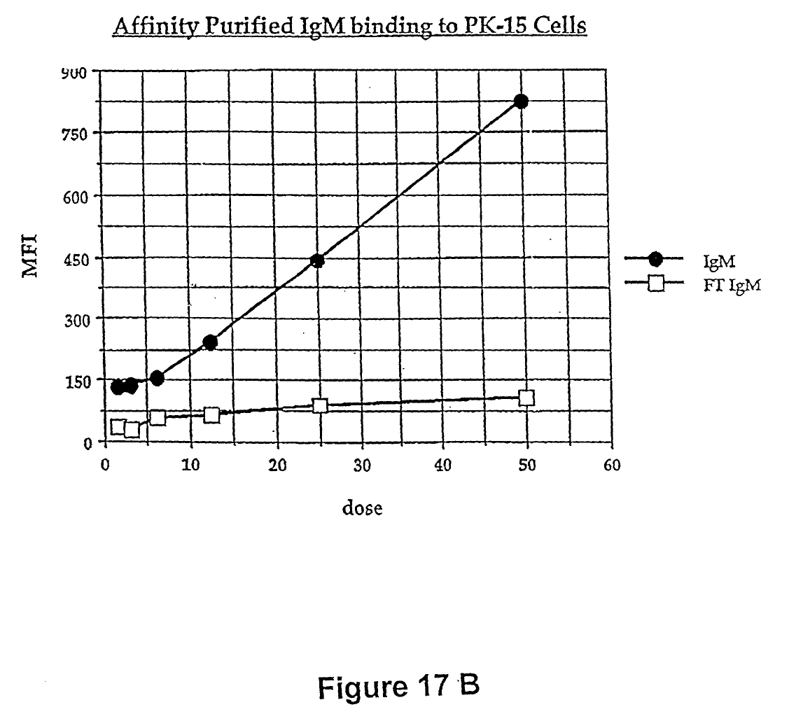

Antibodies. Blood was drawn from healthy normal volunteers. Plasma was separated by centrifugation and allowed to clot. Fibrin was removed and plasma was used immediately or stored in aliquots at −70° C. Rhesus monkey serum (California Regional Primate Research Center, Davis, Calif.) was obtained from blood drawn into vacutainer tubes and allowed to clot. After serum was separated by centrifugation, it was pooled, aliquoted and stored at −20° C. or −70° C. In some experiments, sera was heat-inactivated at 56° C. for 30 minutes to destroy complement hemolytic activity. Antibodies to the αGal epitope were affinity purified on an αGal-Sepharose column, which was prepared by coupling αGal-SH to maleimide-Sepharose (Pierce) through Michael addition chemistry at 10 mg/mL resin. Up to 20 mL pooled NHS (or NMS) normal monkey serum was applied to a 2 mL volume of packed αGal-Sepharose. After the flow through was collected, the column was washed with 10-20 column volumes or until A₂₈₀ reached baseline values and eluted with 0.1 M triethanolamine, pH 11.5 into tubes containing 1 M Tris, pH 8.0. The column was immediately washed with 10-20 volumes of phosphate-buffered saline (PBS). Fractions were assessed for protein concentration by Bradford assay. Peak fractions were pooled and dialyzed against PBS. Affinity-purified anti-αGal Ig was negatively selected for IgG by purification over an MBP column (Pierce, Rockford, Ill. which removed IgM anti-αGal antibodies. IgM anti-αGal was negatively selected by purification over a protein G-Sepharose column (Boehringer Mannheim, Indianapolis, Ind.) which removed IgG anti-αGal antibodies. An elution profile of anti-αGal Ig from an (Gal affinity column is shown in FIG. 16.

SDS-PAGE and Immunoblot Analysis. Antibody-containing fractions and pools were resolved by 4-12% SDS-PAGE (Novex, San Diego, Calif.). Proteins were electrophoretically transferred to PROTRAN™ pure nitrocellulose membranes (Schleicher and Schuell, Keene, N.H.) using XCELL II™ Blot Module blot system (Novex). Membranes were blocked with 2% non-fat dry milk (NFDM) in PBS and probed with anti-human IgG or IgM coupled to alkaline phosphatase (Jackson ImmunoResearch, West Grove, Pa.) or anti-monkey IgG or IgM coupled to alkaline phosphatase (Advanced Chem Tech, Louisville, Ky.). Second step antibody was developed with Western Blue Stabilized Substrate for alkaline phosphatase (Promega Corporation, Madison, Wis.).

ELISA for anti-αGal antibodies. αGal-SH was coupled to maleimide-BSA (bovine serum albumin) (Pierce) at a 2:1 ratio (w/w) according to manufacturer's protocol. The ratio of αGal molecules coupled per BSA molecule was 10-12:1 or 25:1. Alternatively, αGal-BSA or αGal-HAS (human serum albumin) was purchased with C3 or C14 linker groups (Dextra, Redding, England). αGal-BSA (100 μl at 5 μg/mL in PBS) was adsorbed onto 96 well plates for 18 hours at 4° C. Plates were blocked with 2% NFDM in PBS at 4° C. for at least 48 hours prior to use. Plates were stable for at least 3 months. New lots of plates were compared with binding efficacy of the original lot using pooled standard serum. Pooled standard sera, individual sera or affinity purified anti-αGal Ig were titered. Serum (100 μL neat—1/256 diluted in HBSA) or affinity-purified anti-αGal IgG (100 μL of serial two-fold dilutions from 2 mg/mL—1 μg/mL in Hank's balanced salt solution without Ca⁺² or Mg⁺² (HBSA)) were incubated in αGal-BSA coated wells for 60 minutes at 20° C. After washing, anti-αGal Ig was developed with predetermined saturating concentrations (100 μL, usually 1:1000 dilution) of anti-monkey or anti-human IgG or IgM coupled to alkaline phosphatase for 60 minutes at 20° C. After washing the wells 5 times with wash buffer (1% Tween 20 in PBS), plates were developed with 100 μL PPMP (phenolphthalein monophosphate) (Sigma) for 5-20 minutes at 20° C. Reactions were stopped by addition of 100 μL 0.2M Na₂HPO₄ and plates read at A₅₅₀ (PowerWave 340 Microplate Spectrophotometer, Bio-Tek, Winooski, Vt).

αgal Conjugates. αGal (galactose (α1, 3, galactose) epitopes were synthesized at a multigram scale as described in Example 1 and were coupled to a well-defined organic platform as described in Example 2.

Competition ELISA. Serum or affinity purified anti-αGal Ig preparations were titered by ELISA and the 50% binding concentration was determined. For serum, the 50% binding point was reached at a serum dilution of ˜1:5 while for affinity-purified Ig, the 50% dilution binding concentration was ˜12.5 μg/mL. Serum or Ig (50 μL) was incubated with an equal volume of HBSA containing inhibitor which was serially diluted in a two-fold manner from 4 mg/mL to 10 μ g/mL or buffer alone for 60 minutes at 20° C. Anti-αGal Ig or serum +E,uns + inhibitor was then added to αGal-BSA-coated plates and Ig binding was assessed as described. Percent inhibition of anti-diGal binding was calculated as follows: [(OD₅₅₀ Ig source+INH)—OD₅₅₀ blank/(OD₅₅₀ Ig source—INH)—OD₅₅blank]×100. (INH=inhibitor)

ELISpot assay. Spleens from normal rhesus monkeys were minced and prepared as single cell suspension using deburred frosted glass slides. Contaminating erythrocytes were hypotonically lysed and mononuclear cells (MNC) isolated by Ficoll-hypaque density gradient centrifugation. MNC (100 μL) were added in serial two-fold dilutions from 10⁴/cells/mL to 5×10² cells/mL in quadruplicate to ELISA plates bearing αGal-BSA or anti-monkey IgG or IgM. Plates were incubated overnight at 37° C. in a humidified atmosphere with 5% CO₂ and then washed. The footprint of secreted anti-αGal Ig bound to αGal-BSA was developed by incubation with goat anti-monkey IgG or IgM coupled to biotin (Advanced ChemTech) for 60 minutes at 37° C. followed by the addition of ExtrAvidin-alkaline phosphatase (Sigma). Alkaline phosphatase substrate B (100 μL at 1:100 dilution, Bio-Rad, Hercules, Calif.) was added and incubation continued overnight at 20° C. Total Ig- producing cells were similarly determined. The footprints were quantified using a Microtek ScanMaker III flat-bed scanner and personal computer utilizing the Image-Pro imaging software (Media Cybernetics, Univ. Rochester Medical School, Rochester, N.Y.). The ratio of anti-αGal IgG- or IgM-producing cells/total IgG- or IgM-producing cells were calculated.

Cytoxicity assays. The porcine kidney epithelial cell line PK- 15 and porcine aortic endothelial cells (PAEC) (ATCC, 10801 University Blvd., Manassas, Va. 20110-2209) were cultured as directed. For the assay, cells were removed with trypsin—EDTA and replated subconfluently in 96 well plates or on coverslips in 24-well plates. While still subconfluent (within 2 days of replating), cells were used in cytotoxicity assays. Neat, complement-sufficient serum was incubated with inhibitor as described for 60 minutes at 4° C. Serum was then added to wells containing subconfluent cells from which medium had been aspirated immediately prior to serum addition. Wells were incubated with serum±inhibitor for 60-90 minutes at 37° C. Wells were rinsed and cell death was visualized using a Live/Dead kit (Molecular Probes, Eugene, Oreg.) and quantified microscopically in 10 high powered fields or by counting 250 cells. For some experiments, cells were non-enzymatically removed from flasks with cell dissociation solution (Sigma) and single cell suspensions prepared. Assays were performed as for adherent cells except that cytoxicity was quantified by flow cytometry on a Becton-Dickinson FACScalibur.

Results

Antigenic activity of Sal epitope

The synthetically prepared αGal epitope (FIG. 2) was antigenically active, as demonstrated by its ability (when coupled to Sepharose) to remove >95% of anti-αGal Ig from normal rhesus monkey or human serum as measured by FACS.

Activity of tetrameric conjugates. We tested the uGal tetrameric platform constructs which included the PITG platform (compound (cpd) 38) and BMTG (cpd 37) platforms as described in Example 2 and found them to be equivalent in their ability to inhibit Ab from binding to the αGal epitope in the ELISA. Cpd 38 (LJP712) bound anti-αGal Ig and inhibited the binding of IgG anti-αGal to the αGal-expressing porcine kidney epithelial cell line PK-15 and to BSA-αGal adsorbed onto ELISA wells at ˜1 mM. The binding of affinity purified IgM anti-αGal was inhibited 1000-fold less well by cpd 38 than was the IgG anti-αGal.

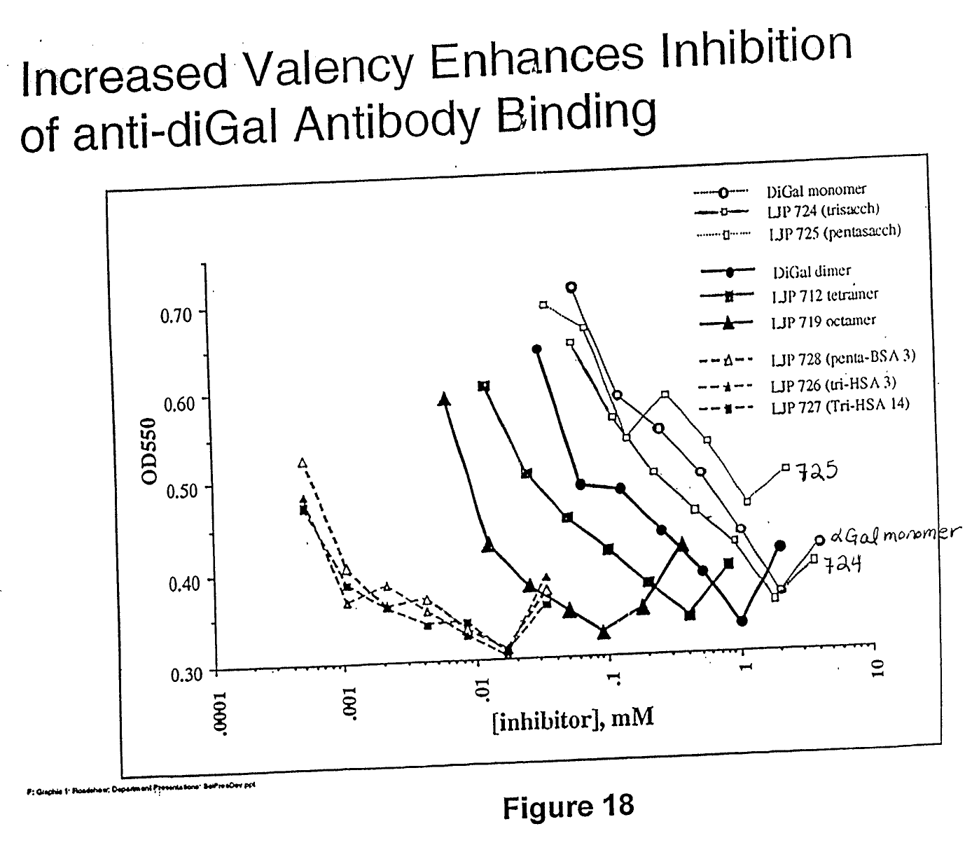

Activity of octameric conjugates. Octameric conjugates (as described in Example 2) were tested. Octameric conjugate cpd 44 (also referred to as LJP 719) was tested in vitro for its ability to inhibit in serum the anti-αGal IgM binding to BSA-αGal. ELISA analysis showed that the αGal epitope when presented on a platform as an octamer inhibited both IgG and IgM anti-αGal in serum from binding to BSA-αGal or to the αGal-expressing porcine kidney epithelial cell line PK-15 and was 5-10 fold more efficient at inhibiting the IgM anti-diGal binding to the diGal epitope, as shown in FIG. 18. This supported our hypothesis that valency of the toleragen was very important in binding the lower affinity IgM molecules.

### Example 5

In vivo evaluation of conjugates

The in vivo efficacy of the toleragens was tested in a dose-escalation study in rhesus monkeys which were treated IV with tetrameric or octameric toleragen or buffer.

Six male rhesus monkeys (3.5-4 kg) were housed at the California Regional Primate Research Center (CRPRC), Davis, Calif. All experimental protocols met CRPRC IACUC (Institutional Animal Care and Use Committee) standards. Monkeys were bled for baseline clinical values and anti-αGal antibody levels. Monkeys were bled for baseline clinical values. Four monkeys were treated IV daily with 2-20 mg/kg of tetravalent platform LJP 712 (cpd 38). Two monkeys received PBS alone intravenously (IV) as a control. Monkeys were bled weekly (5 mL) immediately prior to the IV injection in those animals treated for 60 days with LJP 712 at 2 mg/kg. When monkeys received 10 mg/kg or 20 mg/kg of tetrameric LJP 712 or 20 mg/kg octameric LJP 920 (cpd 46), treatment was for 5-7 days and animals were bled every 2-3 days for 3 mL. Serum samples were analyzed by ELISA for anti-αGal IgG and IgM. In one experiment, two monkeys were treated IV daily for 10 days with 20 mg/kg octameric platform LJP 719. In another experiment, monkeys were treated daily IV with octameric LJP 920 at 20 mg/kg.

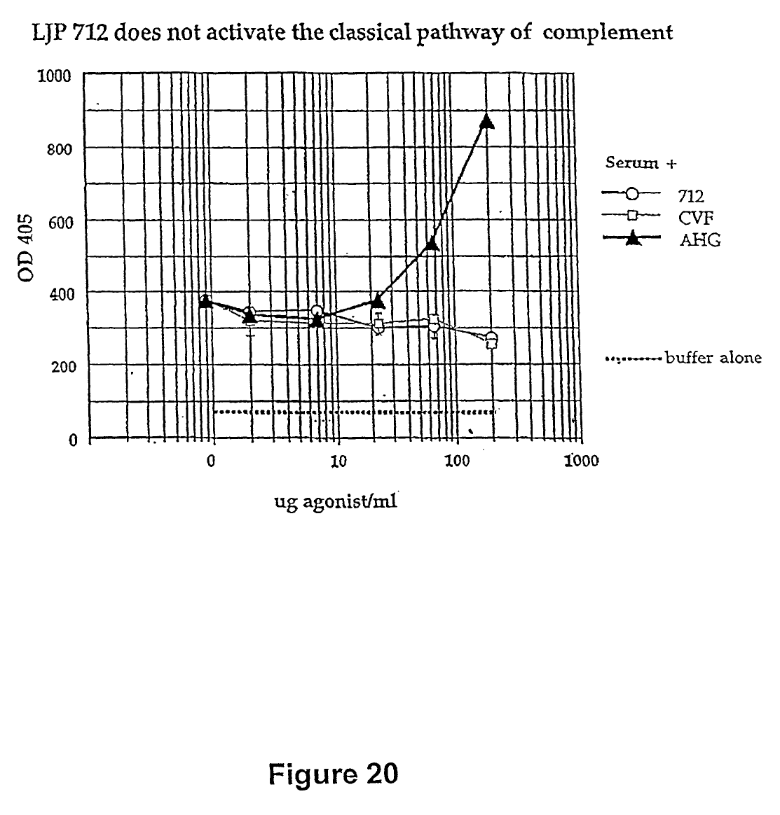

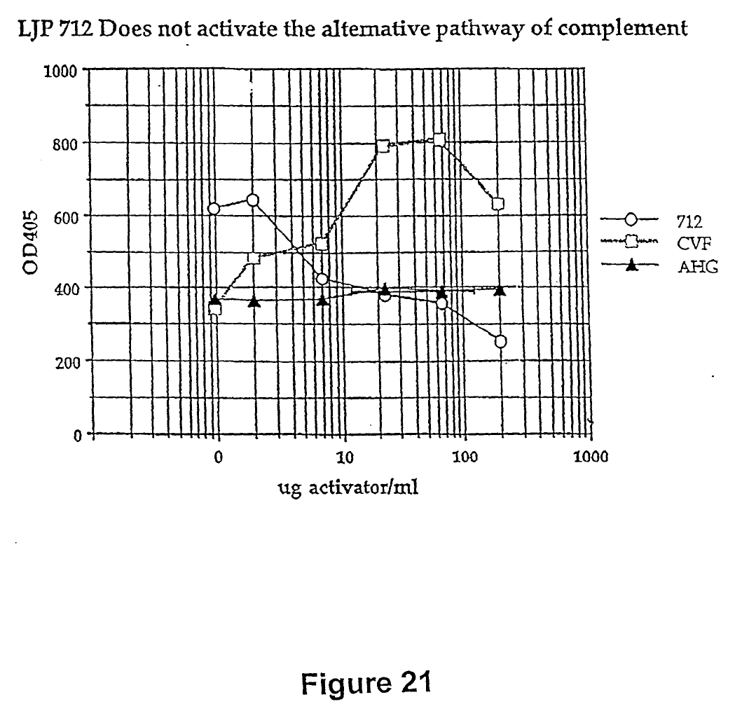

Tetrameric conjugates. To test the in vivo efficacy of LJP 712, the tetravalent toleragen at doses high enough to create a molar excess of toleragen to anti-αGal antibody in the plasma based on 1% of circulating antibody being specific for αGal was used to treat monkeys. Monkeys were treated IV daily with 2 mg/kg of LJP 712 (n=4) or buffer (PBS) (n=2) for 60 days as described above. Blood was drawn weekly and serum tested by ELISA for IgG and IgM anti-αGal. LJP 712 was well-tolerated and did not activate either the classical or alternative complement pathways in vitro, as shown in FIGS. 20 and 21, respectively. There was no statistically significant diminution of anti-αGal Ig responses.

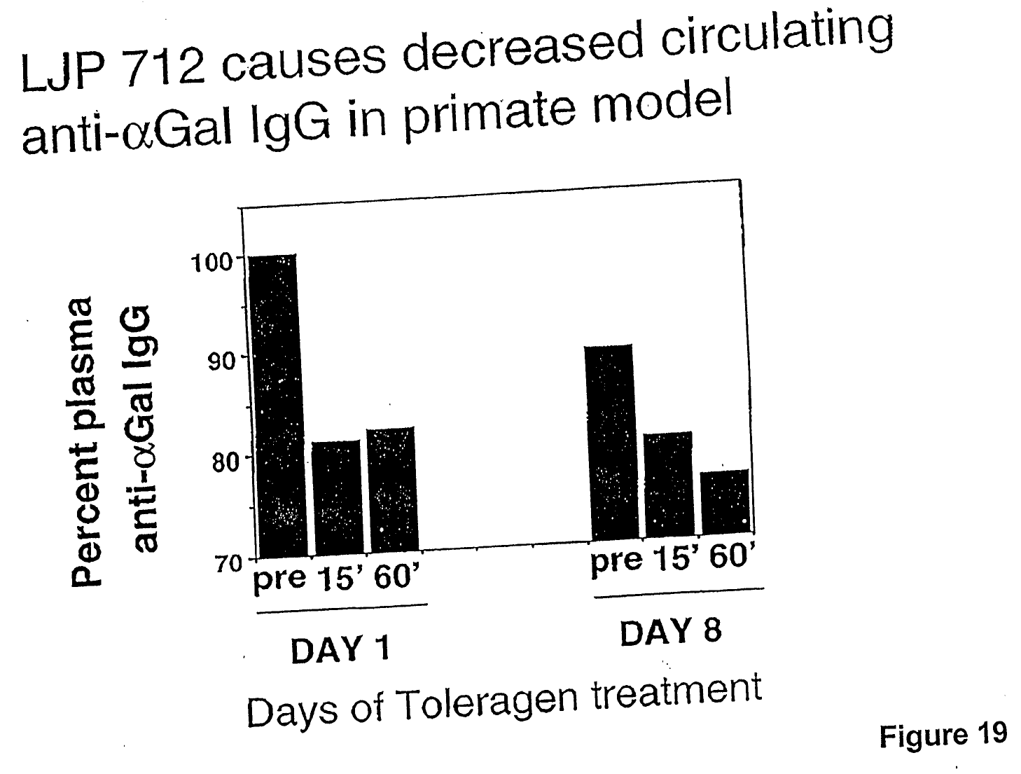

We next sought to determine whether higher doses of LJP 712 were able to effect clearance of anti-αGal Ab from the circulation using shorter term IV dosing modalities. When the dose was increased to 10 mg/kg LJP 712, little diminution of either IgG or IgM anti-αGal was observed. By contrast, daily IV treatment with 20 mg/kg LJP 712 resulted in the diminution of the anti-αGal IgG response by up to 24% p<0.05) by day 8 of treatment and anti-αGal IgM levels by up to 12% (p=NS), as shown in FIG. 19.

Octameric conjugates

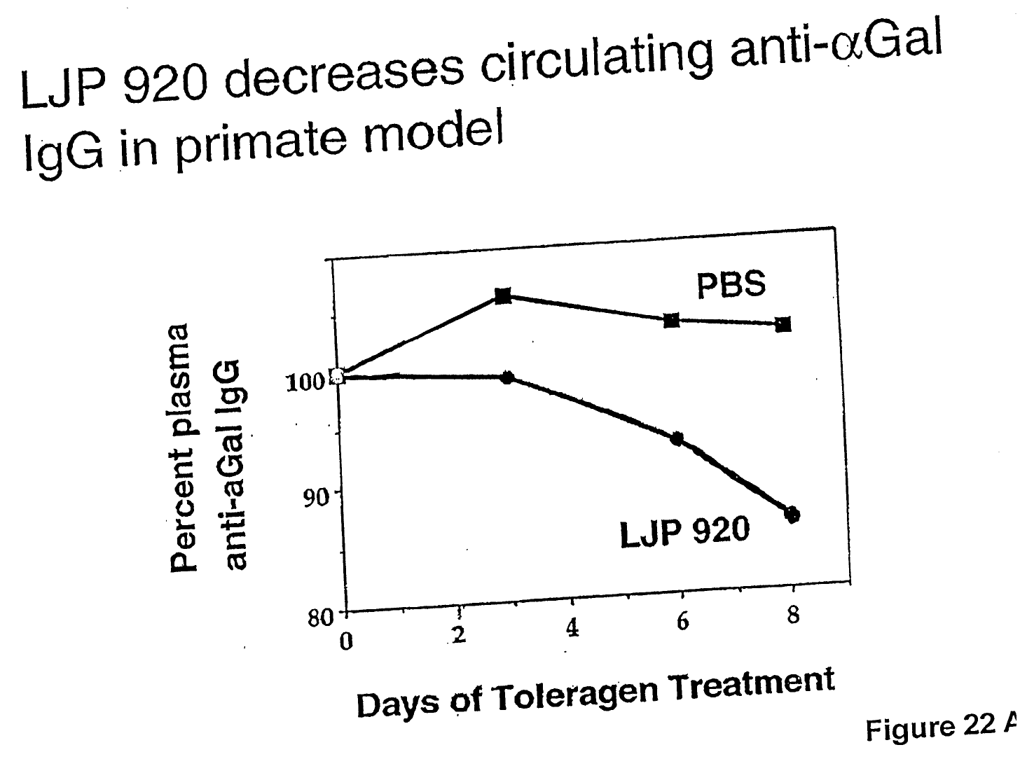

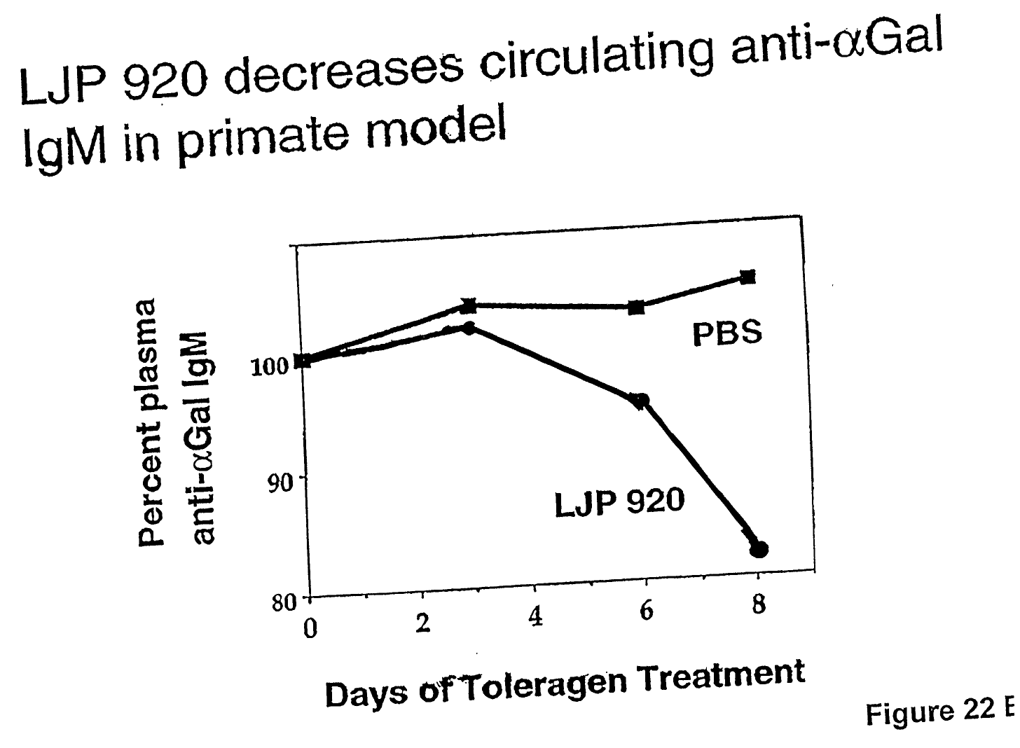

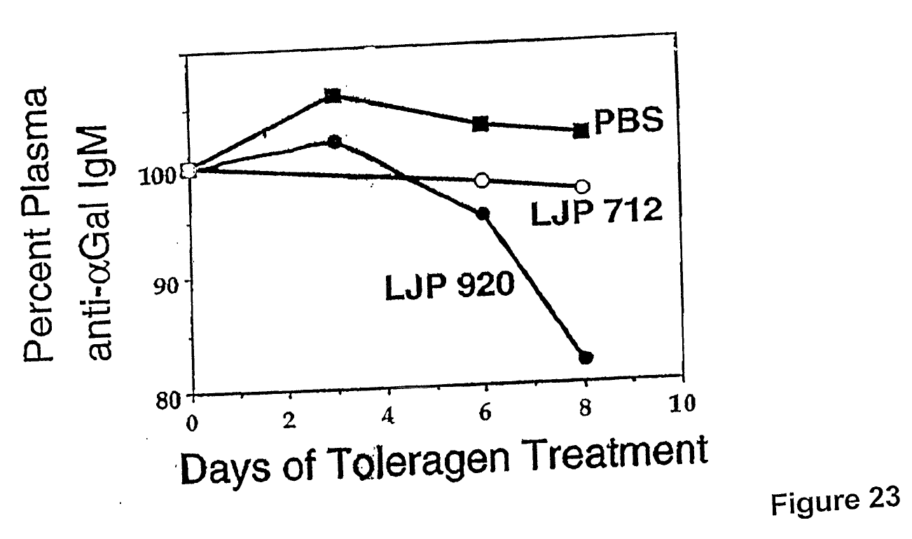

We next determined whether treatment of rhesus monkeys for 7 days with the octameric toleragen LJP 920 led to a diminution in serum levels of anti-αGal Ab. Two monkeys were treated IV daily with PBS and two were treated IV daily with LJP 920 (cpd 46) at 20 mg/kg, a dose which for tetrameric toleragen had shown a diminution in circulating IgG anti-αGal but not IgM anti-αGal. Serum samples were prepared from blood drawn immediately prior to drug or control administration on day 0 (prebleed) and on days 3 and 6 (24 hours post-drug administration). Serum was also prepared on day 8, 24 hours after the last dose with no subsequent dosing administered. LJP 920 (cpd 46) was well-tolerated in the treated animals with no untoward effects as observed by veterinary staff. At day 8, IgG anti-αGal levels were decreased by 11%, similar to the levels seen with tetramer. Control animals showed little change (FIG. 22A). Similarly, there was a diminution of 18% in IgM anti-αGal levels in one monkey and 5% in the replicate animal. By contrast, IgM anti-αGal levels in the control animals did not change in one animal and increased in the replicate animal, as shown in FIG. 22B. That the octamer is more efficient than tetramer at clearing IgM anti-αGal is shown in FIG. 23. These data show that increased valency results not only in a more efficacious molecule in vitro but also in vivo.

Although the foregoing invention has been described in some detail by way of illustration and example for purposes of clarity of understanding, it will be apparent to those skilled in the art that certain changes and modifications will be practiced. Therefore, the description and examples should not be construed as limiting the scope of the invention, which is delineated by the appended claims.

What is claimed is:

1. A method for reducing levels of circulating disease-associated antibodies in an individual, comprising administering to the individual an effective amount of an epitope-presenting carrier.

2. The method of claim 1, wherein the epitope-presenting carrier comprises a valency platform molecule.

3. The method of claim 2, wherein a population of the valency platform molecules has a substantially monodisperse molecular weight.

4. A method of reducing levels of disease-associated antibodies in an individual, comprising treating the individual's blood extracoporeally with an eptiope-presenting carrier under conditions that permit the antibodies to bind the epitope; removing antibody-eptiope-presenting carrier complexes, if any; and returning the blood to the individual.

5. The method of claim 4, wherein the epitope-presenting carrier comprises a valency platform molecule.

6. The method of claim 5, wherein a population of the valency platform molecules has a substantially monodisperse molecular weight.
